SEQUENCE LISTING (8 sequences)

SEQ ID NO: 1 - DNA, 54 bp, synthetic construct
  misc_feature 1..54: note = RNA_S5XX primer
  misc_difference 54: note = n is a, c, g, or t
  source 1..54: mol_type = other DNA
gaggcgtagtggcttagatcgcttttttttttttttttttttttttttttttvn

SEQ ID NO: 2 - DNA, 20 bp, synthetic construct
  misc_feature 1..20: note = 5' biotin modified TSO primer
  misc_feature 1: note = 5' end biotin chemical linkage
  source 1..20: mol_type = other DNA
  misc_feature 1..17: note = DNA
  misc_feature 18..20: note = RNA
tctccgactcagtacatggg

SEQ ID NO: 3 - DNA, 57 bp, synthetic construct
  misc_feature 1..57: note = RNA_N7XX primer
  misc_difference 26..43: note = n is a, c, g, or t
  source 1..57: mol_type = other DNA
aagcagtggtatcaacgcagagtacnnnnnnnnnnnnnnnnnngaggcgtagtggct

SEQ ID NO: 4 - DNA, 47 bp, synthetic construct
  misc_feature 1..47: note = DNA_N7XX primer
  misc_difference 25..32: note = n is a, c, g, or t
  source 1..47: mol_type = other DNA
caagcagaagacggcatacgagatnnnnnnnngtctcgtgggctcgg

SEQ ID NO: 5 - DNA, 51 bp, synthetic construct
  misc_feature 1..51: note = DNA_S5XX primer
  misc_difference 30..37: note = n is a, c, g, or t
  source 1..51: mol_type = other DNA
aatgatacggcgaccaccgagatctacacnnnnnnnntcgtcggcagcgtc

SEQ ID NO: 6 - DNA, 66 bp, synthetic construct
  misc_feature 1..66: note = DDR_PCR_P5 primer
  source 1..66: mol_type = other DNA
aatgatacggcgaccaccgagatctacacgcctgtccgcggaagcagtggtatcaacgcagagtac

SEQ ID NO: 7 - DNA, 20 bp, synthetic construct
  misc_feature 1..20: note = 5' biotin modified TSO primer
  misc_feature 1: note = 5' end biotin chemical linkage
  source 1..20: mol_type = other DNA
  misc_feature 1..17: note = DNA
  misc_feature 18..20: note = RNA
tctccgactcagtacatggg

SEQ ID NO: 8 - DNA, 47 bp, synthetic construct; identical to SEQ ID NO: 4
  misc_feature 1..47: note = Standard Illumina P7 adaptor
  misc_difference 25..32: note = n is a, c, g, or t
  source 1..47: mol_type = other DNA

(19) United States
(12) Patent Application Publication
(10) Pub. No.: US 2026/0317755 A1
(43) Pub. Date: Oct. 15, 2026

(54) METHODS FOR SIMULTANEOUS AMPLIFICATION OF DNA AND RNA

(71) Applicant: BOARD OF REGENTS, THE UNIVERSITY OF TEXAS SYSTEM, Austin, TX (US)

(72) Inventors: Nicholas NAVIN, Houston, TX (US); Kaile WANG, Houston, TX (US); Rui YE, Houston, TX (US)

(21) Appl. No.: 19/111,077

(22) PCT Filed: Sep. 13, 2023
(86) PCT No.: PCT/US2023/074051

§ 371 Date: Mar. 12, 2025

Related U.S. Application Data
(60) Provisional application No. 63/375,485, filed on Sep. 13, 2022.

(51) Int. Cl. C12N 15/10; C12Q 1/6806; C12Q 1/6855; C12Q 1/6869

(52) U.S. Cl. CPC C12N 15/1065; C12N 15/1096; C12Q 1/6806; C12Q 1/6855; C12Q 1/6869

(57) ABSTRACT

The present disclosure provides methods for co-amplifying RNA and DNA from a biological sample in a single reaction compartment. In some aspects, allowing for simultaneously generating RNA and DNA sequencing libraries. In particular, the disclosed methods allow high-throughput amplification and sequencing of both RNA and DNA from a single sample source without the need to divide the sample to separate nucleic acids from each other.

### CROSS-REFERENCE TO RELATED APPLICATIONS

This International Application claims the priority benefit of U.S. Provisional Application No. 63/375,485, filed on Sep. 13, 2022, which is incorporated herein by reference in its entirety.

### REFERENCE TO A SEQUENCE LISTING SUBMITTED ELECTRONICALLY VIA EFS-WEB

This application includes a Sequence Listing submitted electronically via EFS-Web (name: “4443_019PC01_Seqlisting_ST26.xml”; size: 15,659 bytes; and created on: Sep. 5, 2023), which is hereby incorporated by reference in its entirety.

### FIELD OF DISCLOSURE

The present disclosure pertains generally to nucleic acid sequencing technology including methods for generating sequencing libraries from the same sample.

### BACKGROUND

Genomic and transcriptomic sequencing (DNA-seq, RNA-seq) have emerged as powerful tools to study biological systems at a genome-wide scale. However, bulk sequencing studies are limited to profiling a large population of cells, often from complex tissues, which only provide an average measurement over the entire population. Such methods can be useful for characterizing how genotypes influence phenotypes within complex tissues such as cancer tissues that are often composed of many genetically-distinct tumor subclones.

To date a major challenge that exists involves sequencing both genomic DNA and mRNA from the same material from low starting inputs, such as single cells. In particular, due to the limited amount of DNA and RNA present in single cells. Recently, several approaches (eg DR-seq, G&T-seq, SiDR-seq and DNTR-seq) have been tested for sequencing DNA and RNA from the same single cell, however, these methods either require the physical separation of DNA and RNA in the same cell prior to amplification or adding the cell barcode to DNA and RNA during amplification, which limits their throughput to small numbers of cells (e.g., 10-384 cells). Additionally, these methods require very long protocols that span multiple days and are associated with high costs.

Therefore, faster, more flexible and highly scalable approaches are needed to for sequencing both genomic DNA and mRNA biological material having low starting inputs, such as single cells.

### BRIEF SUMMARY

Certain aspects of the disclosure are directed to a method of co-amplifying genomic DNA (gDNA) and RNA from a single biological sample, the method comprising: a) lysing a biological sample to release a plurality of nucleic acids comprising both gDNA and RNA from the biological sample; b) fragmenting the gDNA in the plurality of nucleic acids; c) attaching a DNA adaptor to the fragmented gDNA from (b) to form a plurality of DNA fragment-adaptor molecules; d) synthesizing complementary DNA (cDNA) from the RNA (e.g., unfragmented RNA) in the plurality of nucleic acids, wherein the synthesizing comprises reverse transcription comprising a reverse transcriptase and an RNA primer, wherein the RNA primer comprises an RNA adaptor which is distinguishable from the DNA adaptor to form a plurality of cDNA-adaptor molecules; and e) co-amplifying the plurality of DNA fragment-adapter molecules and the plurality of cDNA-adaptor molecules in the same reaction compartment, wherein one or more barcode sequences are added to the DNA-adaptor and cDNA-adaptor during amplification to form a plurality of DNA amplicons and a plurality of cDNA amplicons; wherein (a)-(e) are performed in the same reaction compartment without physically separating the DNA from the RNA or cDNA. (See, e.g., FIG. 1). In some aspects, the synthesizing in (d) can be performed concurrently with, before, or after (b) or (c). In some aspects, RNA is not fragmented during the fragmenting of the gDNA in (b).

In some aspects, the biological sample is selected from the group consisting of a plurality of cells, a single cell, an organoid, a tissue, a body fluid, naked nucleic acids, and any combination thereof. In some aspects, the biological sample is from an animal, plant, bacterium, fungus, protist, archaeon, or virus.

In some aspects, the biological sample is a plurality of cells. In some aspects, the biological sample is a single cell. In some aspects, the single cell or the plurality of cells comprise a eukaryotic cell or a prokaryotic cell. In some aspects, the biological sample comprises a genetically aberrant cell, cancer cell, or rare blood cell. In some aspects, the single cell or the plurality of cells comprise a genetically engineered cell, an antibody attached single cell, a prelabelled single cell, or a barcoded single cell (See, e.g., FIG. 2). In some aspects, the single cell or plurality of cells comprise a human cell. In some aspects, the single cell or plurality of cells comprise a live cell, a genetically engineered cell, a perturbed cell, or a fixed cell. In some aspects, the biological sample comprises a micro-dissected tissue (e.g., fresh or fixed). In some aspects, the micro-dissected tissue is fresh. In some aspects, the micro-dissected tissue is fixed. In some aspects, the biological sample is from a biopsy. In some aspects, the biological sample is from a surgery sample. In some aspects, the body fluid is blood, urine, saliva, mucus, semen, vaginal fluid, amniotic fluid, cerebrospinal fluid, or a tissue fluid.

In some aspects, the lysing comprises enzymatic lysing, chemical lysing, mechanical lysing, acoustic lysing, electrical-based lysing, or any combination thereof. In some aspects, the lysing in (a) further comprises adding an RNase inhibitor.

In some aspects, the attaching of the DNA adapter in (c) comprises tagmentation. In some aspects, the tagmentation comprises adding a transposase (e.g., Tn5 transposome). In some aspects, the transposase (e.g., Tn5 transposome) is activated or inhibited following the attaching the DNA adapter in (c).

In some aspects, the attaching of the DNA adapter in (c) comprises DNA ligation.

In some aspects, the attaching of the DNA adapter in (c) comprises random sequence extension or PCR.

In some aspects, the RNA primer comprises a poly T tail sequence.

In some aspects, the RNA primer comprises a random sequence. See, e.g., FIG. 3.

In some aspects, template switch oligonucleotides (TSOs) are added during the synthesizing in (d) to form a second end of the cDNA-adaptor molecule.

In some aspects, the order of (b-c) and (d) can occur in reverse, such as the synthesizing and forming the plurality of cDNA-adaptor molecules in (d) occurs prior to the fragmenting in (b) and the attaching and forming the plurality of DNA fragment-adaptor molecules in (c).

In some aspects, steps of (b-c) and (d) can occur concurrently.

In some aspects, (d) is performed during, before, or after (a).

In some aspects, the synthesizing in (d) occurs first, e.g., the reverse transcription occurs prior to lysing the cells in (a). See, e.g., FIG. 4.

In some aspects, the DNA primer and/or the RNA primer comprises a barcode sequence for distinguishing cells. In some aspects, the DNA primer and/or the RNA primer comprise two, three, four, or more different barcode sequences.

In some aspects, the DNA barcode comprises two, three, four, or more different combinatorial barcodes.

In some aspects, the DNA barcode is assigned by tagmentation, PCR or a combination of tagmentation and PCR.

In some aspects, the DNA barcode is assigned by ligation, tagmentation, PCR or the combination of two or more of ligation, tagmentation, and PCR.

In some aspects, two or more combinatorial barcodes are assigned all at the 3′ end, all at the 5′ end, or in a combination of the 3′ and 5′ ends of the DNA or RNA. See, e.g., FIG. 5 and FIG. 6.

In some aspects, the RNA barcode comprises two, three, four, or more different combinatorial barcodes.

In some aspects, the RNA barcode is assigned by reverse transcription, PCR, or the combination of reverse transcription and PCR.

In some aspects, the RNA barcode is assigned by reverse transcription, ligation, PCR, or the combination of reverse transcription, ligation, and PCR.

In some aspects, two or more RNA barcodes are assigned all at the 3′ end, all at the 5′ end, or at both the 3′ and 5′ ends. See, e.g., FIG. 7 and FIG. 8.

In some aspects, the DNA primer and/or the RNA primer comprise a unique molecular identifier (UMI) for distinguishing molecules.

In some aspects, the RNA primer comprises a modification, a label, and/or a detectable label used to separate the cDNA from the DNA.

In some aspects, the modification, the label, and/or the detectable label comprise(s) a biotin modification used to separate the cDNA from the DNA.

In some aspects, the co-amplification comprises polymerase chain reaction (PCR).

In some aspects, the reaction compartment comprises a test tube, a well, a micro-well, a nano-well or a chip array. In some aspects, the reaction compartment comprises a plurality of reaction compartments, optionally a plurality of test tubes, a plurality of wells, a plurality of micro-wells, a plurality of nano-wells or a plurality of chip arrays. In some aspects, the plurality reaction compartments comprising the plurality of DNA amplicons and the plurality of cDNA amplicons are pooled.

In some aspects, the method further comprises (f) separating the plurality of DNA amplicons from the plurality of cDNA amplicons after co-amplification. In some aspects, the plurality of DNA amplicons are separated from the plurality of cDNA amplicons using fragment size, biotin labels, and/or adapter sequence features.

In some aspects, the method further comprises (g) sequencing the plurality of DNA amplicons and the plurality of cDNA amplicons. In some aspects, the sequencing comprises paired-end sequencing or single-read sequencing. In some aspects, the sequencing comprises next-generation sequencing (NGS), single molecule sequencing, or nanopore sequencing. In some aspects, the method comprises identifying a mutation in the DNA or RNA. In some aspects, the mutation is an insertion, a deletion, or a substitution. In some aspects, the mutation is a single nucleotide variation. In some aspects, the mutation is a structural variant. In some aspects, the mutation is associated with a phenotype of interest. In some aspects, the method comprises detecting genomic copy number variation. In some aspects, the method further comprises performing transcriptome quantification or isoform analysis. In some aspects, the method comprises production of cDNA. In some aspects, the method comprises production of a cDNA library. In some aspects, an amplified full-length cDNA library is used to prepare a 3′ RNA-seq library.

### BRIEF DESCRIPTION OF DRAWINGS

FIG. 1 shows an example workflow of a “wellDR-seq” method. Dispensed single cell or low input material of each tube/well is first lysed to release the DNA and RNA (step1); then DNA adaptors are added into the gDNA fragments (using tagmentation reaction with Tn5 transposome in this example protocol) (Step 2); and RNA adaptors are added during reverse transcription and template switching occurs in this protocol (Step 3). DNA and cDNA (RNA) with their specific adaptors are then amplified through the subsequent PCR reaction. The primers used for amplifying DNA and RNA can have barcode sequences (e.g. bc1-bc4 as shown), which are used as the well/tube/cell barcode for labeling DNA and RNA molecules from the same well/tube/cell (step 4). To each one of the modalities (e.g., RNA), a pre-amplification step that specifically enriches that modality can be performed in this protocol. Barcoded DNA and RNA molecules from all reaction tubes/wells are then pooled together (step 5). DNA and RNA libraries can be separated according fragment sizes, biotin modifications or other methods, followed by preparing the pooled DNA and RNA sequencing libraries according to the research purpose and sequencing instruments. In this example, the DNA and RNA libraries are first separated by fragment size. The RNA library is further enriched by streptavidin beads capture, since a biotin modification primer is used to specifically label the RNA. The enriched RNA library can further be enriched by amplifying with RNA library specific primers. The amplified full-length cDNA (as depicted) then is used to prepare 3′ RNA-seq library. To guarantee that the cell barcodes can be sequenced and determined, all of the cell barcodes of one modality must be either in the sequencing read or the sequencing index reads. In this example, for the RNA modality, bc3 and bc4 are in sequencing read1, while for DNA modality, bc1 and bc2 are in sequencing index read1 and index read2.

FIG. 2 shows exemplary input cells compatible with the disclosure. Input cells can be genetically engineered cells, antibody labelled cells or barcoded cells.

FIG. 3 shows an exemplary flow diagram of a method of the disclosure including profiling DNA and total RNA using random primers for reverse transcription.

FIG. 4 shows an exemplary flow diagram of a method of the disclosure using a single cell/nucleus suspension. A single cell/nucleus suspension is first used to perform an in situ reverse transcription reaction when the cell/nucleus remains intact. Then, the single cell/nucleus is dispensed into a single tube/well. Next, lysis and tagmentation are performed to fragment DNA and add DNA adaptors. Subsequently, the DNA and cDNA are coamplified in the same reaction vessel. Barcoded DNA and RNA of each single tube/well are then mixed together (shown as step 5), and DNA and RNA are separated. Finally the RNA and DNA libraries are enriched individually to prepare sequencing libraries.

FIG. 5 shows an exemplary flow diagram of a method of the disclosure. To assign a cell barcode to the DNA of each single cell, the transposome itself has the barcode. This barcode together with the PCR primer barcodes added to the DNA, are used to achieve combinatorial indexing of the single cells.

FIG. 6 shows an exemplary flow diagram of a method of the disclosure. To assign the cell barcode to the DNA of each single cell, the combinatorial barcodes are assigned to the same end of the DNA molecule, such as the 3′end.

FIG. 7 shows an exemplary flow diagram of a method of the disclosure. To assign the cell barcode to RNA (cDNA) molecules, the combinatorial barcodes (bc3 and bc4) are labelled at the 5′ ends of the RNA molecules by two rounds of PCR reactions.

FIG. 8 shows an exemplary flow diagram of a method of the disclosure. The combinatorial barcodes (bc3 and bc4) are labelled at both ends of the RNA.

FIG. 9 shows an exemplary flow diagram of a method of the disclosure. Single cell DNA mutations and RNA expression are measured with wellDR-seq. The method can be combined with DNA capture panels with target regions, or whole exome sequencing of the scDNA library with wellDR-seq.

FIG. 10 shows an exemplary flow diagram of a method of the disclosure. Using cells labelled with polyA based oligonucleotides (like lipid-based or antibody based sample multiplexing) or labelled by genetic engineered cells (such as CRISPR-Cas9) as input. WellDR-seq can be used to profile DNA, RNA and the cell labels together.

FIG. 11 shows an exemplary flow diagram of a method of the disclosure. Using cells labelled with DNA adaptors, WellDR-seq can profile DNA, RNA and the cell labels concurrently.

FIGS. 12A-12B show low-throughput single tube experiments of 12 cells for single cell DNA copy number profiling and RNA expression analysis. FIG. 12A shows copy number profiles from 12 single cells profiled for the wellDR-seq method using single tube compartments. Each row represents the copy number profile from a single cell, with Log 2 segment ratios showing copy number gains (red) and losses (blue). FIG. 12B shows quality control metrics for RNA expression profiles from the same 12 single cells depicted in FIG. 12A.

FIGS. 13A-13G show mid-throughput for a wellDR-seq method using 384 well plates to profile breast cancer cells (from MDA-MB-231 cell line). FIG. 13A is a uniform manifold approximation and projection (UMAP) depicting two clusters identified by the single cell gene expression data. FIG. 13B shows the top 10 differential expressed gene between cluster 0 and 1 from the RNA expression data. FIG. 13C shows a UMAP of RNA profiles with annotations showing the plates from which the cells were profiled. FIG. 13D shows Pearson correlation of gene expression (MDA-MB-231 cell line) detected by wellDR-seq (WDR) and 3′DE-seq (Takara). FIG. 13E shows Pearson correlation of gene expression (MDA-MB-231 cell line) detected by wellDR-seq (WDR) 10× Genomics' single cell 3′ RNA-seq (tenx). FIG. 13F shows DNA superclones mapped to the UMAP of the RNA high-dimensional space. FIG. 13G shows a heatmap of DNA copy number aberrations from the scDNA data, in which superclones and subclones were based on the heatmap clustering results. Left side bar shows the plates that each cell was sequenced from. RNA_Cluster side-bar shows the RNA clustering results using gene expression profiles from FIG. 13A.

FIGS. 14A-14G show result from a high-throughput nanowell based wellDR-seq method of the MDA-MB-231 cancer cell line. FIG. 14A is a UMAP showing two clusters of single cells from MDA-MB-231 identified by gene expression data. FIG. 14B shows the top 10 differential expressed genes between cluster 0 and 1. FIG. 14C shows gene (nFeature_RNA), UMI (nCount_RNA) and mitochondrial percentages of the two RNA clusters. FIG. 14D shows Pearson correlation of gene expression from MDA-MB-231 detected by wellDR-seq (WDR) and 3′DE-seq (Takara). FIG. 14E shows Pearson correlation of the gene expression data from the MDA-MB-231 cell line detected by wellDR-seq (WDR) 10× Genomics' single cell 3′ RNA-seq (tenx). FIG. 14F shows single cell DNA data of superclones mapped to the RNA UMAP high-dimensional space. FIG. 14G shows a heatmap of DNA copy number aberrations in single cells according to the DNA data, with superclones and subclones annotated based on the heatmap clustering results. While the RNA Cluster side-bar shows the RNA clustering results using the gene expression profiles from FIG. 14A.

### DETAILED DESCRIPTION OF THE DISCLOSURE

Disclosed herein is are methods (referred to herein as “wellDR-seq”) comprising simultaneously co-amplifying DNA and RNA from a single biological sample in one compartment (e.g., cell/sample/well) and does not require physically separating the nucleic acids (DNA/RNA) from low input materials or single cells prior to performing amplifications and constructing sequencing libraries for sequencing.

The methods disclosed herein are highly scalable while allowing for amplification of DNA and RNA simultaneously from thousands of individual cells/samples/wells. This technology can include assigning cell barcodes to DNA and RNA in the same input materials or single cells during the amplification procedures. In some aspects, the amplified DNA and RNA with cell barcodes from different input materials or single cells can then be pooled together, followed by preparing only two (DNA and RNA) sequencing libraries separately thereafter. The methods disclosed herein are compatible with low-throughput (e.g., single tube reactions), mid-throughput (e.g., multiple tube-based (e.g., 8-strip tubes) reactions or 96/384-well plate reactions) and high-throughput (e.g., nanowell or microwell) platforms. The methods disclosed herein are applicable in biomedicine and research including fields such as cancer, pre-natal genetic diagnosis, developmental biology and clinical diagnostics, particularly, where it is necessary to link genotypic and phenotypic data together to understand complex biological processes and human diseases.

The nucleic acids (DNA and RNA) used in the disclosed methods can be obtained from any source. Sources of nucleic acid molecules include, but are not limited to, organelles, cells (single cells or plurality of cells), tissues, organs, and organisms. In some aspects, the DNA and the RNA are from a single cell or a selected population of cells. In some aspects, the DNA and RNA are obtained from a biological sample comprising a eukaryotic cell (e.g., animal, plant, fungus, or protist), prokaryotic cell (e.g., bacterium or archaeon), or a virus. For example, the biological sample can comprise a genetically aberrant cell, cancer cell, or rare blood cell. In one aspects, the cell is a human cell. In some aspects, the DNA and RNA are obtained from a sample of micro-dissected tissue or a biopsy.

Non-limiting examples of the various aspects are shown in the present disclosure.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

The term “isolated” is meant, when referring to a polypeptide, that the indicated molecule is separate and discrete from the whole organism with which the molecule is found in nature or is present in the substantial absence of other biological macro-molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein.

The term “DNA” refers to genomic DNA (gDNA), chromosomal DNA, plasmid DNA, phage DNA, or viral DNA that is single stranded or double stranded. DNA can be obtained from prokaryotes or eukaryotes.

The term “genomic DNA” or “gDNA” is used interchangeably with chromosomal DNA.

The term “messenger RNA” or “mRNA” refers to an RNA that is without introns and that can be translated into a polypeptide.

The term “complementary DNA” or “cDNA” refers to a DNA that is complementary or identical to an mRNA, in either single stranded or double stranded form.

As used herein, “polymerase” and its derivatives, generally refers to any enzyme that can catalyze the polymerization of nucleotides (including analogs thereof) into a nucleic acid strand. Typically but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some aspects, the second polypeptide can include a reporter enzyme or a processivity-enhancing domain. Optionally, the polymerase can possess 5′ exonuclease activity or terminal transferase activity. In some aspects, the polymerase can be optionally reactivated, for example through the use of heat, chemicals or re-addition of new amounts of polymerase into a reaction mixture. In some aspects, the polymerase can include a hot-start polymerase or an aptamer based polymerase that optionally can be reactivated.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

As used herein, the terms “ligating,” “ligation,” and their derivatives refer generally to the act or process for covalently linking two or more molecules together, for example, covalently linking two or more nucleic acid molecules to each other. In some aspects, ligation includes joining nicks between adjacent nucleotides of nucleic acids. In some aspects, ligation includes forming a covalent bond between an end of a first and an end of a second nucleic acid molecule. In some aspects, for example aspects wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some aspects, any means for joining nicks or bonding a 5′phosphate to a 3′ hydroxyl between adjacent nucleotides can be employed. In an exemplary embodiment, an enzyme such as a ligase can be used. Generally for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some aspects, the ligase includes an enzyme capable of catalyzing the joining of nicks between adjacent nucleotides of a nucleic acid. In some aspects, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

The term “amplicon” refers to the amplified product of a nucleic acid amplification reaction, e.g., RT-PCR or PCR.

The terms “reverse-transcriptase PCR” and “RT-PCR” refer to a type of PCR where the starting material is mRNA. The starting mRNA is enzymatically converted to complementary DNA or “cDNA” using a reverse transcriptase enzyme. The cDNA can be used as a template for a PCR reaction.

Polymerase chain reaction (PCR) generally refers to a method used to rapidly make (amplify) millions to billions of copies (complete or partial) of a DNA sample.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nucl. Acids Res., 28, e63, 2000), each of which is hereby incorporated by reference in its entirety.

As used herein, the terms “amplify” and “amplification” refer to enzymatically copying the sequence of a polynucleotide, in whole or in part, so as to generate more polynucleotides that also contain the sequence or a complement thereof. The sequence being copied is referred to as the template sequence. Examples of amplification include DNA-templated RNA synthesis by RNA polymerase, RNA-templated first-strand cDNA synthesis by reverse transcriptase, and DNA-templated PCR amplification using a thermostable DNA polymerase. Amplification includes all primer-extension reactions. Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

The term “hybridize” refers to a sequence specific non-covalent binding interaction with a complementary nucleic acid. Hybridization can occur to all or a portion of a nucleic acid sequence. Those skilled in the art will recognize that the stability of a nucleic acid duplex, or hybrids, can be determined by the Tm. Additional guidance regarding hybridization conditions can be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, Vol. 3, 1989.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. In some aspects, primers within the scope of the disclosure include orthogonal primers, amplification primers, constructions primers and the like. Pairs of primers can flank a sequence of interest or a set of sequences of interest. Primers and probes can be degenerate in sequence. Primers within the scope of the present disclosure bind adjacent to a target sequence. A “primer” can be considered a short polynucleotide, generally with a free 3′—OH group that binds to a target or template potentially present in a sample of interest by hybridizing with the target, and thereafter promoting polymerization of a polynucleotide complementary to the target. In some aspects, primers of the instant disclosure are comprised of nucleotides ranging from 17 to 30 nucleotides. In some aspects, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

As used herein, “incorporating” a sequence into a polynucleotide refers to covalently linking a series of nucleotides with the rest of the polynucleotide, for example at the 3′ or 5′ end of the polynucleotide, by phosphodiester bonds, wherein the nucleotides are linked in the order prescribed by the sequence. A sequence has been “incorporated” into a polynucleotide, or equivalently the polynucleotide “incorporates” the sequence, if the polynucleotide contains the sequence or a complement thereof Incorporation of a sequence into a polynucleotide can occur enzymatically (e.g., by ligation or polymerization) or using chemical synthesis (e.g., by phosphoramidite chemistry).

As used herein, the term “adapter”, “adaptor”, “adapter sequence”, or “adaptor sequence” refers to short oligonucleotides that can be ligated to one or both ends of a DNA fragment of interest, e.g., so that the DNA can be combined with primers for amplification. In some aspects, adapters can be added to the 5′ and/or 3′ end of a DNA fragment. In some aspects, the adapter sequence can include barcoding sequences, forward/reverse primers (e.g., for paired-end sequencing) and the binding sequences for immobilizing the DNA fragments (e.g., to the flowcell and allowing bridge-amplification).

As used herein, the terms “label” and “detectable label” refer to a particle, ion, isotope, small molecule, macromolecule, molecular complex, or other suitable material capable of use for detection, including, but not limited to, radioactive isotopes, fluorescers, chemiluminescers, chromophores, enzymes, enzyme substrates, enzyme cofactors, enzyme inhibitors, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which can be used in the practice of the methods disclosed herein include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′,4′,5′,7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, NADPH, horseradish peroxidase (HRP), and α-β-galactosidase.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

As used herein, the term “biological sample” or “sample” typically refers to a sample obtained or derived from one or more biological sources (e.g., a tissue or organism or cell culture) of interest, as described herein. In some aspects, a source of interest comprises an organism, such as an animal or human. In some aspects, a source of interest comprises a microorganism, such as a bacterium, virus, protozoan, or fungus. In some aspects, a source of interest may be a synthetic tissue, organism, cell culture, nucleic acid or other material. In some aspects, a sample can be a multi-organism sample (e.g., a mixed organism sample). In some aspects, a sample can comprise a cell, a plurality of cells, a cell mixture a tissue sample, or a tissue mixture.

As used herein, the term “tagmentation” refers to the modification of DNA by a transposome complex comprising transposase enzyme and transposon end sequence in which the transposon end sequence further comprises adaptor sequence. Tagmentation results in the simultaneous fragmentation of the DNA and ligation of the adaptors to the 5′ ends of both strands of duplex fragments. As used herein, the term “transposase” refers to an enzyme which can catalyze a tagmentation reaction. In some aspects, the transposase is bound to a substrate polynucleotide prior to tagmentation. In some aspects, the transposase comprises Tn5. In some aspects, the transposase comprises an engineered transposase.

As used herein, the term “transpososome” refers to a complex comprising a transposase bound to a substrate polynucleotide. In some aspects, a transpososome comprises a multimer of two or more transposase subunits. In some aspects, the substrate polynucleotide comprises a tag. In some aspects, the substrate polynucleotide comprises a barcode. In some aspects, the barcode comprises a combinatorial barcode.

As used herein, the term “combinatorial barcode” refers to a polynucleotide sequence which, when combined with one or more additional combinatorial barcodes, results in one or more sequences which, in combination and in a given arrangement, allow for unique identification of an attached polynucleotide of interest. In some aspects, two polynucleotides of interest are considered uniquely barcoded if they contain the same two or more combinatorial barcodes, wherein the two combinatorial barcodes are in a different 5′ to 3′ order or arrangement relative to each other or the polynucleotide of interest to which the combinatorial barcodes are attached. For example, combinatorial barcodes A, B, and C, having different sequences, are arranged in the 5′ to 3′ order of ABC and attached to a first polynucleotide of interest at the 5′ end; for a second polynucleotide of interest, the same sequences A, B, and C are attached and arranged in the 5′ to 3′ order of ACB at the 5′ end. In this example, the first and second polynucleotides of interest are uniquely identifiable despite comprising the same different sequences A, B, and C, based on the arrangement of A, B, and C. In another example, a first polynucleotide of interest and a second polynucleotide of interest are each tagged with combinatorial barcodes A, B, and C, arranged in the 5′ to 3′ order, but the combinatorial barcodes are attached to the 5′ end of the first polynucleotide, and are attached to the 3′ end of the second polynucleotide. In this example, the first and second polynucleotides of interest are uniquely identifiable despite comprising the same different sequences A, B, and C, arranged in the same order, based on the arrangement ABC relative to the first or second polynucleotide of interest.

### II. Biological Material

Certain aspects of the disclosure are directed to a methods that comprise co-amplifying DNA (e.g., gDNA) and RNA from a single biological sample, wherein the method comprises lysing a biological sample to release a plurality of nucleic acids comprising both DNA and RNA from the biological sample.

The input material (e.g., biological sample) for the methods disclosed herein can be or can include single cell/nucleus or other low input materials, for example: several cells/nuclei or multiple cells/nuclei, which can be processed, modified, fixed, tagmented or antibody attached to the cells/nuclei. In some aspects, the input material (e.g., biological sample) can be or can include organoids, small portions/piece of tissues, methanol-fixed or formalin-fixed, paraffin-embedded (FFPE) tissue samples, blood drops, buffy coat, body fluids, swabs, naked DNA/RNA, etc.

In some aspects, to co-amplify and achieve the preparation DNA and RNA libraries simultaneously, the method comprises releasing DNA and RNA from input material (e.g., the biological sample) in a reaction compartment. In some aspects, the release procedure (e.g., lysis) can use an enzyme-based method (e.g., a protease); a chemical-based method (e.g., a detergent such as Tween-20, Triton X-100, or a combination thereof); mechanical, acoustic, or electrical based method; or any combination thereof. In some aspects, this release (e.g., lysis) step can break the cellular/nuclear membrane and digest the chromatin structures to expose the DNA and RNA.

In some aspects, an RNase inhibitor can be added at this step to help prevent or reduce RNA from degradation during lysis.

In some aspects, the biological sample is obtained or derived from one or more biological sources (e.g., a tissue or organism or cell culture) of interest, as described herein. In some aspects, a source of interest comprises an organism, such as an animal or human. In some aspects, a source of interest comprises a microorganism, such as a bacterium, virus, protozoan, or fungus. In some aspects, a source of interest may be a synthetic tissue, organism, cell culture, nucleic acid or other material. In some aspects, a sample can be a multi-organism sample (e.g., a mixed organism sample). In some aspects, a sample can comprise a cell mixture or a tissue mixture. In some aspects, the sample can include fetal DNA. In some aspects, a biological sample can be isolated DNA or other nucleic acids. In some aspects, a biological sample is or comprises biological tissue or fluid. In some aspects, the biological tissue or fluid can include bone marrow; blood; blood cells; stem cells, ascites; tissue samples, biopsy samples or or fine needle aspiration samples; cell-containing body fluids; free floating nucleic acids; protein-bound nucleic acids, riboprotein-bound nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as a ductal lavages or broncheoalveolar lavages; vaginal fluid, aspirates; scrapings; bone marrow specimens; tissue biopsy specimens; fetal tissue or fluids; surgical specimens; feces, other body fluids, secretions, and/or excretions; and/or cells therefrom, etc. For example, in some aspects, a biological sample can be obtained by methods selected from the group consisting of biopsy (e.g., fine needle aspiration or tissue biopsy), surgery, collection of body fluid (e.g., blood (or plasma or serum separated therefrom), lymph, feces etc.), etc. In some aspects, the sample can be processed (e.g., by removing one or more components of and/or by adding one or more agents to a primary sample). In some aspects, the sample can be fixed tissues (e.g. FFPE tissues, methanol fixed or formalin-fixed tissues).

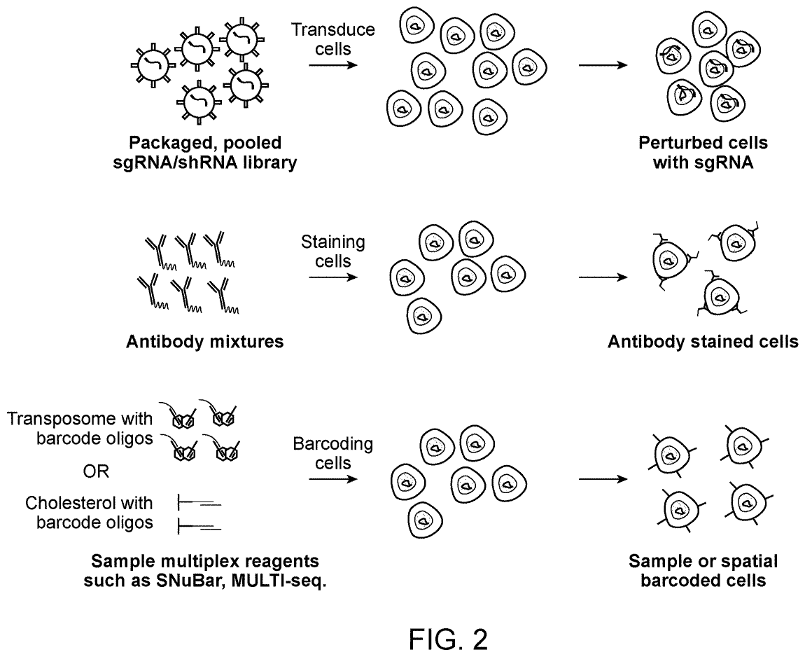

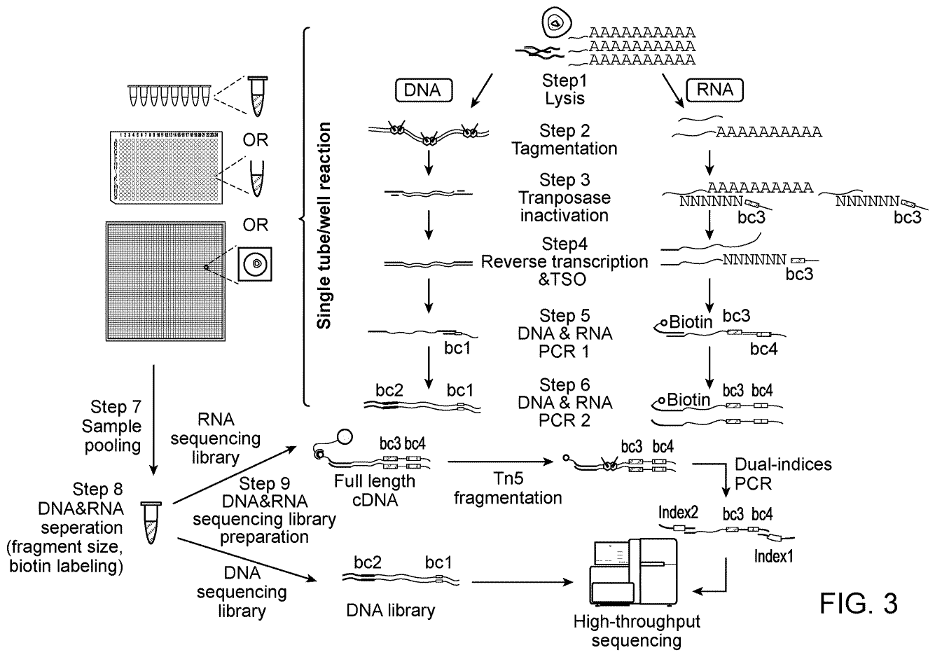

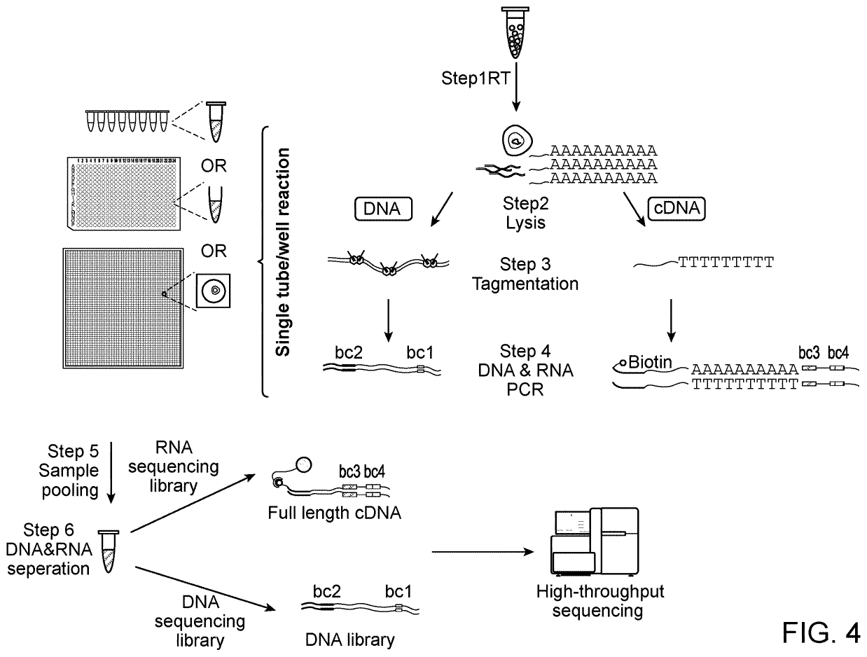

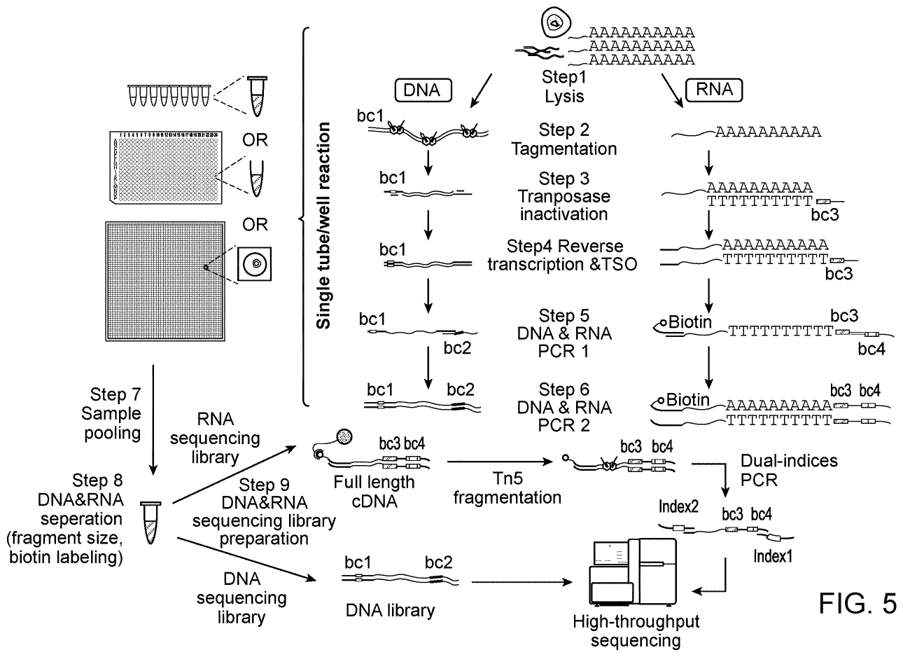

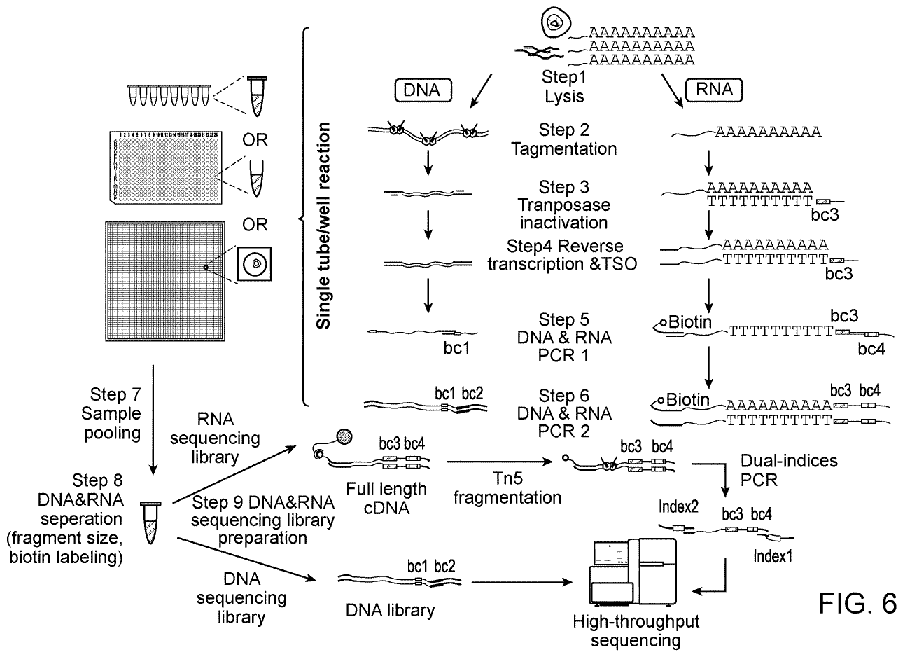

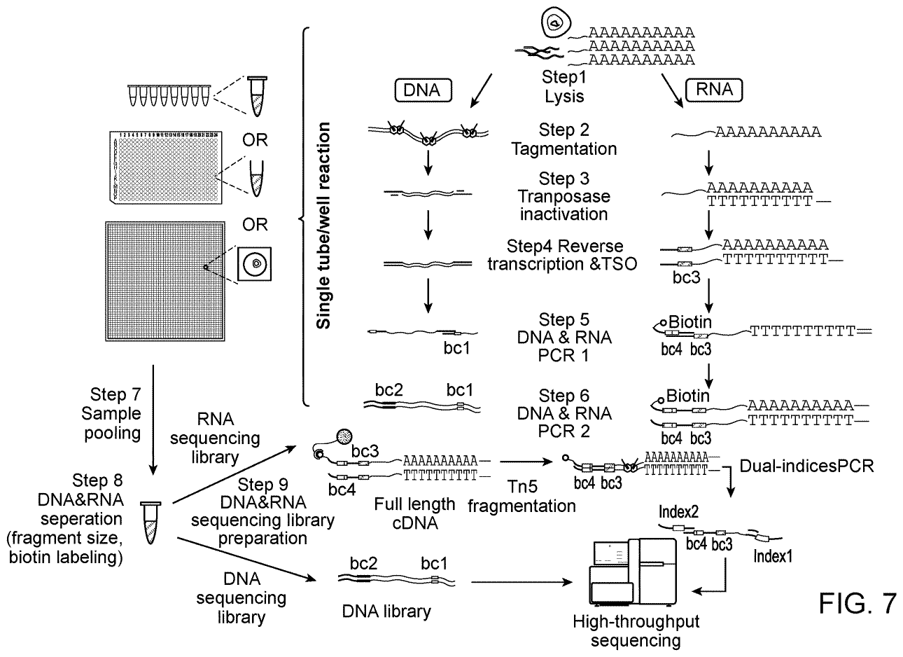

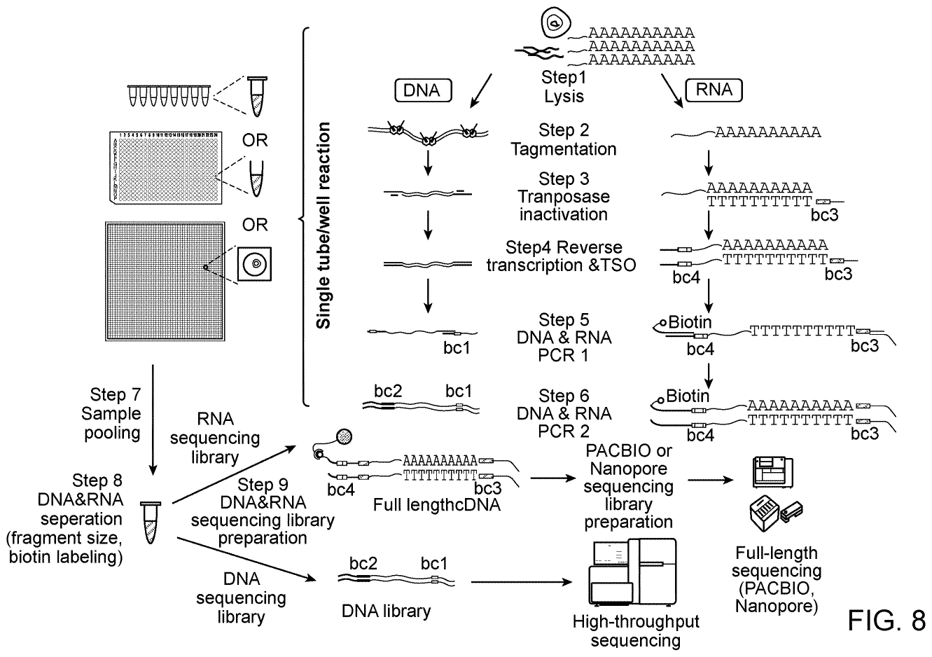

In some aspects, the biological sample is a single cell. In some aspects, the cell is a eukaryotic cell or a prokaryotic cell. In some aspects, the biological sample is from an animal, plant, bacterium, fungus, protist, archaeon, or virus. In some aspects, the biological sample comprises a genetically aberrant cell, cancer cell, or rare blood cell. In some aspects, the single cell or the plurality of cells comprise a genetically engineered cell, an antibody attached single cell, a prelabelled single cell, or a barcoded single cell (e.g., see FIG. 2). In some aspects, the cell is a human cell. In some aspects, the cell is a live cell, a genetically engineered cell, a perturbed cell (such as using CRISPR/CAS9 to perform multi-locus gene perturbation as described in Perturb-seq (Dixit et. al, Cell, 2016)), and/or a fixed cell. In some aspects, the DNA and the RNA are from a sample of micro-dissected tissue. In some aspects, the DNA and the RNA are from a biopsy.

### III. Preparing and Adding Adaptors to DNA

Certain aspects of the disclosure are directed to a method of co-amplifying DNA and RNA from a single biological sample, the method comprising: lysing a biological sample to release a plurality of nucleic acids comprising both gDNA and RNA from the biological sample; fragmenting the DNA; and attaching a DNA adaptor to the fragmented DNA from to form a plurality of DNA fragment-adaptor molecules. In some aspects, RNA is not fragmented during the fragmenting of the DNA.

In some aspects, the DNA is fragmented into shortened fragments or sections of DNA. In some aspects, an adaptor is added (e.g., ligated to) the fragmented DNA. In some aspects, the fragmenting of the DNA comprises contacting the nucleic DNA with a transposase. In some aspects, the transposase is a Tn5 transposase. In some aspects, the Tn5 transposase is EZTn5TM, NexteraV2, or TS-Tn5059. Selective fragmentation and tagging of DNA (i.e., tagmentation) can be accomplished by treatment of the nucleic acids with a Class 2 transposase. Preferably, the transposase is hyperactive to allow efficient tagmentation. Hyperactive Tn5 transposases can be used in the practice of the methods of the disclosure and are commercially available from a variety of sources, including Illumina Inc. (San Diego, Calif.), Creative Biogene (Shirley, N.Y.), Epicentre Biotechnologies (Madison, Wis.), and Mandel Scientific (Ontario, Canada). Oligonucleotide adapters are complexed with the transposase to generate an active transposome. Transposome units insert randomly into a genomic template resulting in concerted fragmentation of the DNA and ligation of adapter oligonucleotide sequences to the generated fragments. The transposable oligonucleotide adapter comprises a common priming site for DNA-specific amplification to allow amplification of the generated DNA fragments using a set of universal DNA-specific primers. For a description of tagmentation and hyperactive transposases useful for carrying out the method, see, e.g., U.S. Pat. Nos. 9,080,211; 9,238,671; 6,294,385; 8,383,345; 9,040,256; 9,074,251; 7,083,980; and 8,829,171; U.S. Patent Application Publication No. 2015/0291942; and Brouilette et al. (2012) Dev. Dyn. 241 (10): 1584-1590; Petzke et al. (2009) Appl. Microbiol. Biotechnol. 83 (5): 979-986; Lyell et al. (2008) Appl. Environ. Microbiol. 74 (22): 7059-7063; Steiniger et al. (2006) Biochemistry 45 (51): 15552-15562; Steiniger-White et al. (2002) J. Mol. Biol. 322 (5): 971-982; Naumann et al. (2002) J. Bacteriol. 184 (1): 233-240; Twining et al. (2001) J. Biol. Chem. 276 (25): 23135-23143; Naumann et al. (2000) Proc. Natl. Acad. Sci. U.S.A. 97 (16): 8944-8949; York et al. (1998) Nucleic Acids Res. 26 (8): 1927-1933; and Goryshin et al. (1998) J. Biol. Chem. 273 (13): 7367-7374; herein incorporated by reference in their entireties.

In some aspects, to label the DNA molecules, a lysate (comprising DNA and RNA from the biological sample) can be used to perform reactions such as tagmentation to fragment and add an adaptor at the same time using a transposome (e.g., Tn5 transposome). In some aspects, the transposome can be commercial and loaded such as the Tn5 transposome (Illumina) with universal oligonucleotides, or the transposome can be assembled by combining the transposase with transposase recognized DNA oligonucleotides (e.g., Mosaic End (ME) sequences). The oligonucleotides that attach to the transposase can have a barcode or part of a barcode sequence included for distinguishing different cells, while the oligonucleotides also serve as the identifier to distinguish the DNA from the RNA molecular in the same input material (e.g., single cell/nuclei), which are in the same compartment. The transposome can be inactivated or inhibited by protein denaturing detergents (e.g., SDS) or heating with EDTA or other inhibitors after the tagmentation reaction. In some aspects, the lysate can be used to perform random priming using the adaptor (primer) sequences with random sequences to generate short DNA fragments, while the adaptor (primer) sequence comprises part of sequence severing as the DNA adaptor, which is distinguishable from the RNA/cDNA adaptor.

The adaptors that are added to the 5′ and/or 3′ end of a nucleic acid (e.g., DNA) can comprise a universal sequence. A universal sequence is a region of nucleotide sequence that is common to, i.e., shared by, two or more nucleic acid molecules. Optionally, the two or more nucleic acid molecules also have regions of sequence differences. Thus, for example, the 5′ adapters can comprise identical or universal nucleic acid sequences and the 3′ adapters can comprise identical or universal sequences. A universal sequence that may be present in different members of a plurality of nucleic acid molecules can allow the replication or amplification of multiple different sequences using a single universal primer that is complementary to the universal sequence.

### IV. Reverse Transcription and Adding RNA/cDNA Adaptors

Certain aspects of the disclosure are directed to a method of co-amplifying DNA and RNA from a single biological sample, the method comprising: lysing a biological sample to release a plurality of nucleic acids comprising both DNA and RNA from the biological sample and synthesizing cDNA from the RNA, wherein the synthesizing comprises reverse transcription comprising a reverse transcriptase and an RNA primer, wherein the RNA primer comprises an RNA adaptor which is distinguishable from the DNA adaptor to form a plurality of cDNA-adaptor molecules.

In some aspects, the RNA can be amplified by reverse transcribing RNA into cDNA with a reverse transcriptase, and then performing PCR (i.e., RT-PCR), as described above. Alternatively, a single enzyme may be used for both steps as described in U.S. Pat. No. 5,322,770, incorporated herein by reference in its entirety. In this manner, cDNA can be generated from all types of RNA, including mRNA, non-coding RNA, microRNA, siRNA, and viral RNA.

In some aspects, to profile the transcriptomics of single cell/nucleus or low input materials, the RNA or cDNA molecules are attached (e.g., ligated) to specific adaptors, which are distinguishable from the DNA adaptors of the disclosure. In some aspects, the RNA molecules are converted into cDNA (complimentary DNA) by reverse transcriptase (e.g., MMLV reverse transcriptase, AMV reverse transcriptase or other RT enzymes). In some aspects, the primer used for reverse transcription (RT) can prime using RNA molecules with poly-T tails, or random sequences, as well as other targeted gene specific sequences. In some aspects, the RNA primer has a universal sequence component (e.g., greater or equal to about 6 base pairs) that can be used as the RNA adaptor (e.g., PCR amplification handle) sequence for amplifying the cDNA in later steps of the method. In some aspects, the primer can comprise a cell barcode or part of a cell barcode allowing for cell identity. In some aspects, the primer can comprise a unique molecular identifier (UMI) sequence to distinguish individual transcripts from PCR duplicate reads. In some aspects, the primer can comprise modifications (such as biotin), which can be used to separate the RNA from DNA molecular afterward co-amplification. In some aspects, the cell barcode can comprise 1 or 2 oligonucleotide sequences, or multiple oligonucleotide sequences.

In some aspects, during the RT reaction, a template switch oligonucleotide (TSO) can be added to form a second end of the cDNA sequences (as with the other RNA adaptor). In some aspect, the TSO sequence(s) can comprise a cell barcode or part of a cell barcode, serving as a cell identifier, and/or can also have a unique molecular identifier (UMI) sequence to distinguish individual transcripts from PCR duplicate reads. In some aspects, the TSO can comprise modifications (such as biotin) used to separate the RNA from DNA molecules after co-amplification.

During lysis or other steps, the RNA molecule can become fragmented or lose the 5′ cap structure. In some aspect, e.g., instead of using the TSO strategies, other methods can be used to add the RNA adaptor (PCR priming) sequences to the second end of the cDNA molecules. In some aspects, the adaptor is added using a ligation reaction at the 3′end of the cDNA; using a second strand synthesis using oligonucleotides with random priming sequences and universal tails or oligonucleotides with target priming sequence and universal tails.

In some aspects, the RNA adaptors (PCR priming) sequences comprise modifications (such as biotin) that can be used to separate the RNA from DNA molecules after the co-amplification reactions.

In some aspects, to facilitate the separation of DNA and RNA after co-amplification, and optional pooling all of the samples (or cells) together and preparation of separate DNA and RNA libraries, the sequences used to label the DNA can comprise at least two distinct sequences (e.g., oligonucleotide attached on ME sequence of the transposome; or the sequence(s) used to label the RNA (e.g., RT primers and/or TSO sequences)).

In some aspects, the RNA primer comprises a poly T tail universal sequences. In some aspects, the RNA primer comprises template switching oligonucleotides. In some aspects, the barcoding comprises using unique sequence identifiers or primer biotinylation.

The adaptors that are added to the 5′ and/or 3′ end of a nucleic acid (e.g., cDNA) can comprise a universal sequence. A universal sequence is a region of nucleotide sequence that is common to, i.e., shared by, two or more nucleic acid molecules. Optionally, the two or more nucleic acid molecules also have regions of sequence differences. Thus, for example, the 5′ adapters can comprise identical or universal nucleic acid sequences and the 3′ adapters can comprise identical or universal sequences. A universal sequence that may be present in different members of a plurality of nucleic acid molecules can allow the replication or amplification of multiple different sequences using a single universal primer that is complementary to the universal sequence.

### V. DNA and RNA/cDNA Pre-Amplification and Co-Amplification

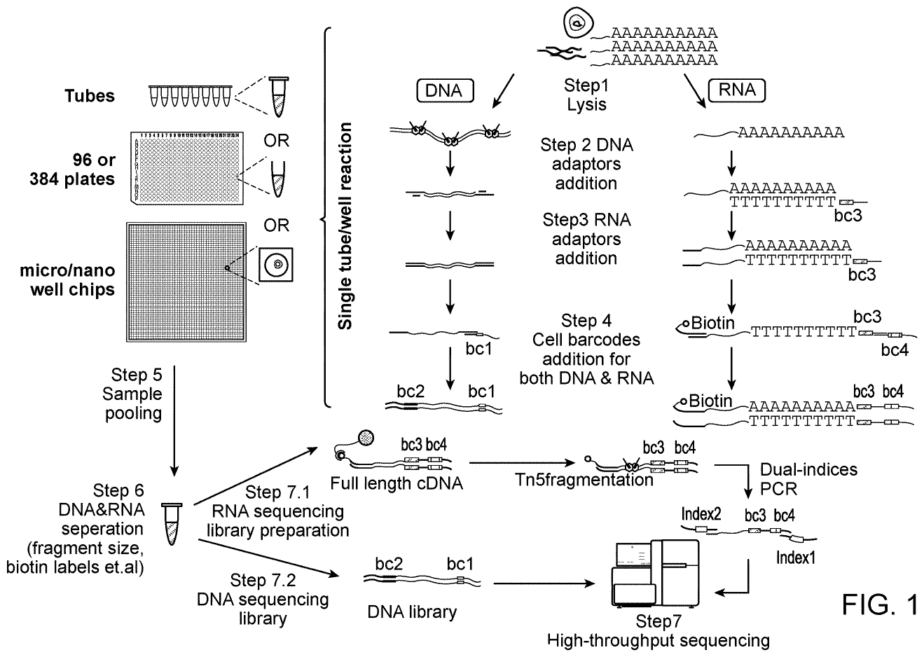

Certain aspects of the disclosure are directed to a method of co-amplifying genomic DNA (gDNA) and RNA from a single biological sample, the method comprising: a) lysing a biological sample to release a plurality of nucleic acids comprising both gDNA and RNA from the biological sample; b) fragmenting the gDNA in the plurality of nucleic acids; c) attaching a DNA adaptor to the fragmented gDNA from (b) to form a plurality of DNA fragment-adaptor molecules; d) synthesizing complementary DNA (cDNA) from the RNA (e.g., unfragmented RNA) in the plurality of nucleic acids, wherein the synthesizing comprises reverse transcription comprising a reverse transcriptase and an RNA primer, wherein the RNA primer comprises an RNA adaptor which is distinguishable from the DNA adaptor to form a plurality of cDNA-adaptor molecules; and e) co-amplifying the plurality of DNA fragment-adapter molecules and the plurality of cDNA-adaptor molecules in the same reaction compartment, wherein one or more barcode sequences are added to the DNA-adaptor and cDNA-adaptor during amplification to form a plurality of DNA amplicons and a plurality of cDNA amplicons; wherein (a)-(e) are performed in the same reaction compartment without separating the DNA from the RNA or cDNA (See, e.g., FIG. 1). In some aspects, the synthesizing in (d) can be performed concurrently with, before, or after (b) or (c). In some aspects, steps of (b-c) and (d) can occur concurrently. In some aspects, step (d) can occur before the cell lysis. In some aspects, step (d) can occur between (a) and (b). In some aspects, RNA is not fragmented during the fragmenting of the gDNA in (b).

### Cell/Material Barcode Addition for DNA and RNA Modalities and DNA and RNA Coamplification Reactions

In some aspects, the methods of the disclosure can use different adaptor sets to label DNA and RNA separately for preamplification of one of the individual assays (e.g., RNA or DNA). In some aspects, to enrich the number of molecules from one of the assays (e.g., RNA assay), both forward and reverse primers of the assay that is desired to be enriched can be added, while only add one of the primers of the other assay (e.g., DNA assay). In some aspects, one or two primers (forward or/and reverse) can be added to the assay that is desired to be enriched, but then primers for the other assay are not added. The one assay enrichment procedure can take place before the DNA and RNA co-amplification step (pre-enrichment) or afterwards (post-enrichment). In some aspects, the primers can comprise a cell barcode or part of a cell barcode serving as a cell identifier, the primers can also have modifications (such as biotin) that are used to separate a pool of RNA from DNA molecules after the co-amplification.

In some aspects, the primer pairs for the DNA assay and the primer pairs for the RNA assay are all added into the same reaction compartment during the co-amplification step. In some aspects, the primers can have cell barcodes or part of cell barcodes serving as the cell identifiers, and the primers can also have modifications (such as biotin) used to separate the RNA from DNA molecules after the co-amplification step.

In some aspect, the annealing temperatures of the co-amplification step can be used to favor one of the assays or both assays to control the total number of molecules amplified. For example, by controlling the favored annealing temperature, the method can be adjusted to balance the DNA and RNA assay product amounts.

### Cell/Material Barcode Addition for DNA and RNA Modalities and DNA and RNA Coamplification Reactions

Certain aspects of the discloser are directed to co-amplification of the DNA (DNA assay) and RNA/cDNA (RNA assay) in a single reaction compartment.

In some aspects, the co-amplification comprises PCR. In some aspects, amplification comprises performing a clonal amplification method, such as, but not limited to bridge amplification, emulsion PCR (ePCR), or rolling circle amplification. In particular, clonal amplification methods such as, but not limited to bridge amplification, emulsion PCR (ePCR), or rolling circle amplification may be used to cluster amplified nucleic acids in a discrete area (see, e.g., U.S. Pat. Nos. 7,790,418; 5,641,658; 7,264,934; 7,323,305; 8,293,502; 6,287,824; and International Application WO 1998/044151 A1; Lizardi et al. (1998) Nature Genetics 19:225-232; Leamon et al. (2003) Electrophoresis 24:3769-3777; Dressman et al. (2003) Proc. Natl. Acad. Sci. USA 100:8817-8822; Tawfik et al. (1998) Nature Biotechnol. 16:652-656; Nakano et al. (2003) J. Biotechnol. 102:117-124; herein incorporated by reference). For this purpose, additional adapter sequences (e.g., adapters with sequences complementary to universal amplification primers or bridge PCR amplification primers) suitable for high-throughput amplification may be added to DNA or cDNA fragments at the 5′ and 3′ ends. For example, bridge PCR primers, attached to a solid support, can be used to capture DNA templates comprising adapter sequences complementary to the bridge PCR primers. The DNA templates can then be amplified, wherein the amplified products of each DNA template cluster in a discrete area on the solid support.

In some aspects, the reaction compartment comprises a test tube, a well, a micro-well, a nano-well or a chip array. DNA and cDNA may be amplified prior to sequencing using any suitable polymerase chain reaction (PCR) technique known in the art. In PCR, a pair of primers is employed in excess to hybridize to the complementary strands of a target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. Typically, the primer oligonucleotides are in the range of between 10-100 nucleotides in length, such as 15-60, 20-40 and so on, more typically in the range of between 20-40 nucleotides long, and any length between the stated ranges.

The polynucleotide sample is denatured, preferably by heat, and hybridized with first and second primers that are present in molar excess. Polymerization is catalyzed in the presence of the four deoxyribonucleotide triphosphates (dNTPs-dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler, e.g., Perkin Elmer.

In some aspects, the single reaction compartment comprises a test tube, a well, a micro-well, a nano-well or a chip array. In some aspects, the single compartment comprises a plurality of single compartments, optionally a plurality of test tubes, a plurality of wells, a plurality of micro-wells, a plurality of nano-wells or a plurality of chip arrays. In some aspects, the plurality single compartments comprising the plurality of DNA amplicons and the plurality of cDNA amplicons are pooled.

In some aspects, before pooling the DNA and RNA fragments from all of the cells/sample together, the DNA or RNA from the same cell/sample are labelled with cell/sample barcodes. The DNA and RNA from the same cell/sample material can be labelled with same barcode sets or different barcode sets (by knowing the DNA/RNA barcode correspondence relationship). The DNA and RNA modalities can be barcoded by single or multiple barcodes, or the combination of barcodes. In some aspects the two barcodes, three barcodes or multiple barcodes (>3) can be located in one end (e.g., 5′ end) of each modality, or alternatively can be located in both ends (e.g., 5′ and 3′) of each modality. In some aspects, the method can use tagmentation based chemistries (e.g., Tn5 transposome) to fragment DNA. The DNA fragments can be barcoded by using tagmentation enzyme with different adaptors (eg. by attaching different oligonucleotide sequences to the mosaic sequences of Tn5 transposase), or can be barcoded in the subsequent PCR steps through PCR primers that contain different barcodes (indices) or barcode combinations, or barcoded by both approaches by combining the barcode introduced in tagmentation step and PCR steps. In some aspects, the barcoding for the RNA assay, can occur at the reverse transcription (RT) step by using different RT primers, or barcoded at the later PCR steps through different PCR primers with different barcodes (indices) or barcode combinations, which can be barcoded by combining the barcode introduced in the reverse transcription step and PCR steps. In order to separate the DNA and RNA libraries after pooling all of the libraries from single cells or sample materials, DNA and RNA libraries in each single cell or low input sample are labeled with different adaptors, which will be used to distinguish these two different libraries after pooling. In some aspects, at least two of these adaptors are different between the DNA and RNA libraries to ensure the procedure of preparing DNA and RNA libraries separately can occur after pooling. In some aspects, one of the adaptors or primers from the DNA or RNA assay can comprise one or multiple modifications (such as biotin) to help separate the two assays after pooling.

### Sample Pooling and Separation

In some aspects, after molecules from DNA and RNA assays of the same cell/material are labelled with cell barcodes, the libraries from all of the reaction wells can be pooled together. From this pool of cells/samples, a physical separation of the amplified DNA and RNA libraries can be performed. In some aspects, the separating methods can be based on the DNA/RNA fragment sizes, can be based on the modifications that are used to label one of the modalities (e.g., Biotin modifications), can be based on different adaptors used to distinguish the DNA and RNA modalities, or can be other methods that can distinguish amplified DNA and RNA molecules.

In some aspects, the method further comprises separating the DNA from the cDNA after co-amplification.

In some aspects, the separating the DNA from the cDNA is based on a molecular feature of the DNA or the cDNA. In some aspects, the molecular feature is fragment size, biotin labels or different adapter sequences.

Certain aspects of this disclosure provide a method that can amplify DNA and RNA from the same low input material (tens, hundreds or thousands of cells) or single cells simultaneously without physically separating the nuclei acids before amplification (the method sometimes referred to here as “wellDR-seq”). In some aspects, the cell barcodes (or material barcodes) are attached to DNA and RNA from the same input material or single cells during amplification. In some aspects, wellDR-seq is compatible with tube-based reactions (eg. single and 8-strip tubes reaction), plate-based reactions (eg. 96 well and 384 well plate) and high density nanowell (eg. thousands of wells). In some aspects, the amplified DNA (genome) from these reactions can be used to detect genome-wide copy number variations, DNA mutations, structural variations and other genomic aberrations, while the amplified RNA (transcriptome) can be used to detect gene expression levels, identify new transcripts, map gene and exon boundaries, identify alternative splicing events and other applications. In some aspects, since the DNA and RNA are measured from the same input material or single cell, wellDR-seq is able to investigate how DNA aberrations impact gene expression, levels and how these two layers of molecular information interact with each other.

In some aspects, wellDR-seq can link genomic information to phenotypes in low input materials, including single cells. This approach is expected to have broad applications in studying genome and transcriptome interactions, how mutations or copy number variations affect the gene expression in normal or tumor cells, and quantifying the gene dosage effects in different types of cells. The wellDR-seq approach can also be used in many research applications to study the basic biology of development, tumorigenesis and cancer progression, to identify predictive and prognostic biomarkers, and identify actionable targets in clinic.

Certain aspects of this disclosure provide two separate libraries for flexible manipulation downstream: a DNA library based on the original DNA and a cDNA library based on the original RNA produced by any of the methods described herein. The DNA library or cDNA library can be sequenced to provide an analysis of gene expression in single cells or in a plurality of single cells. The amplified DNA or cDNA library can be sequenced and analyzed using methods known to those of skill in the art, e.g., by next-generation sequencing (NGS). In certain exemplary aspects, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLID, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

Aspects of the disclosure also provide methods for analyzing gene expression in a plurality of single cells, the method comprising the steps of preparing a cDNA library using the method described herein and sequencing the cDNA library. Any of the polynucleotide sequences described herein can be used to identify larger fragments or full-length coding sequences of the gene with which they are associated. Methods of isolating larger fragment sequences are known to those of skill in the art.

The cDNA library can be sequenced by any suitable screening method. In particular, the cDNA library can be sequenced using a high-throughput screening method, such as Applied Biosystems' SOLID sequencing technology, or Illumina's Genome Analyzer. In some aspects of the disclosure, the cDNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

The DNA or gDNA library generated by the methods disclosed herein can be useful for, but not limited to, DNA variant detection, copy number analysis, fusion gene detection and structural variant detection. The cDNA library generated by the methods disclosed herein can be useful for, but not limited to, RNA variant detection, gene expression analysis, and fusion gene detection. The DNA and cDNA libraries can also be used for paired DNA and RNA profiling.

The expression profiles described herein are useful in the field of predictive medicine in which diagnostic assays, prognostic assays, pharmacogenomics, and monitoring clinical trials are used for prognostic (predictive) purposes to thereby treat an individual prophylactically. Accordingly, some aspects relate to diagnostic assays for determining the expression profile of nucleic acid sequences (e.g., RNAs), in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary aspects, methods of diagnosing and/or prognosing one or more diseases and/or disorders using one or more of expression profiling methods described herein are provided.

### VI. Sequencing

Certain aspects of the disclosure are directed to a method of sequencing DNA and/or RNA librarities from co-amplifying genomic DNA (gDNA) and RNA from a single biological sample, the method comprising: a) lysing a biological sample to release a plurality of nucleic acids comprising both gDNA and RNA from the biological sample; b) fragmenting the gDNA in the plurality of nucleic acids; c) attaching a DNA adaptor to the fragmented gDNA from (b) to form a plurality of DNA fragment-adaptor molecules; d) synthesizing complementary DNA (cDNA) from the RNA (e.g., unfragmented RNA) in the plurality of nucleic acids, wherein the synthesizing comprises reverse transcription comprising a reverse transcriptase and an RNA primer, wherein the RNA primer comprises an RNA adaptor which is distinguishable from the DNA adaptor to form a plurality of cDNA-adaptor molecules; and e) co-amplifying the plurality of DNA fragment-adapter molecules and the plurality of cDNA-adaptor molecules in the same reaction compartment, wherein one or more barcode sequences are added to the DNA-adaptor and cDNA-adaptor during amplification to form a plurality of DNA amplicons and a plurality of cDNA amplicons; wherein (a)-(e) are performed in the same reaction compartment without separating the DNA from the RNA or cDNA; (f) separating the DNA amplicons from the cDNA amplicons after co-amplification; and (g) sequencing the DNA amplicons and/or the cDNA amplicons, wherein the DNA barcodes are used to identify DNA sequences and the RNA barcodes are used to identify RNA sequences. In some aspects, RNA is not fragmented during the fragmenting of the gDNA in (b).

In some aspects, after the step of separating the DNA amplicons and RNA/cDNA amplicon modalities is completed, the DNA and/or RNA can be used for high-throughput sequencing platforms if the PCR primers used to amplify the DNA modality are the same sequencing adaptor that is used for sequencing. For example, if the Illumina Tn5 transposome (eg TDE1) is used during the tagmentation step, and the PCR primers used to amplify DNA modality are Nextera PCR forward and reverse primers, then after separation of the DNA and RNA, the DNA library is ready-to-load for sequencing libraries.

In some aspects, the separated DNA and/or RNA amplification products can be used to prepare different sequencing libraries, e.g., according to the research purpose and sequencing instrument requirements. In some aspects, the separated DNA and/or RNA amplification products can also be used for further enrichment using DNA or RNA specific adaptors added during the previous steps. In some aspects, further enriched products can be used as input materials for performing high throughput sequencing platforms if the PCR primers used to amplify the DNA modality are the same sequencing adaptor used for the sequencing reactions. In some aspects, these methods can be modified to prepare different sequencing libraries (e.g., according to the research purpose and sequencing instrument requirements).

In some aspects, high throughput sequencing can be performed. In some aspects, the high throughput sequencing platforms include, but are not limited to, next generation sequencing, single molecule sequencing and nanopore sequencing.

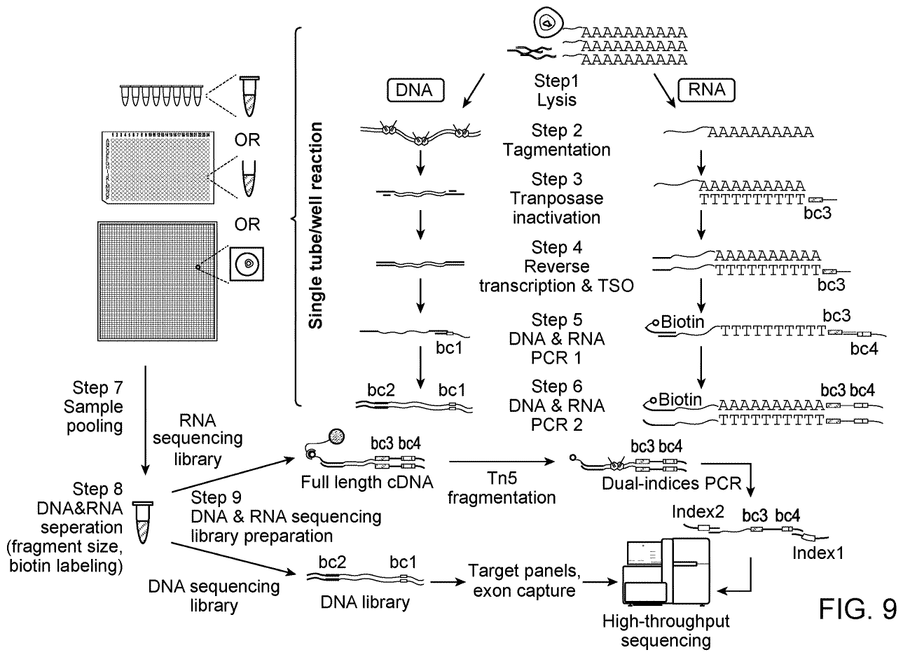

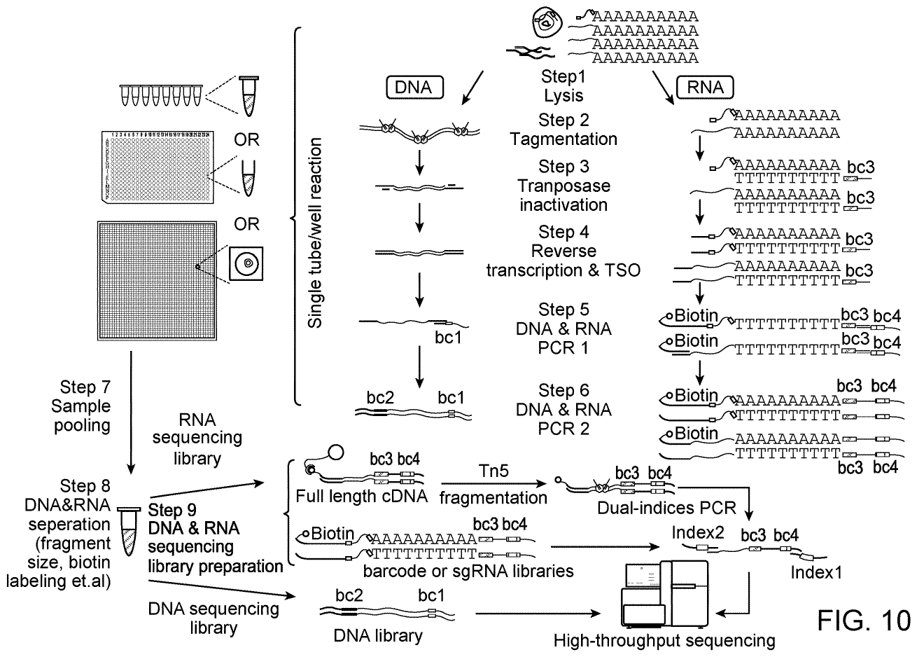

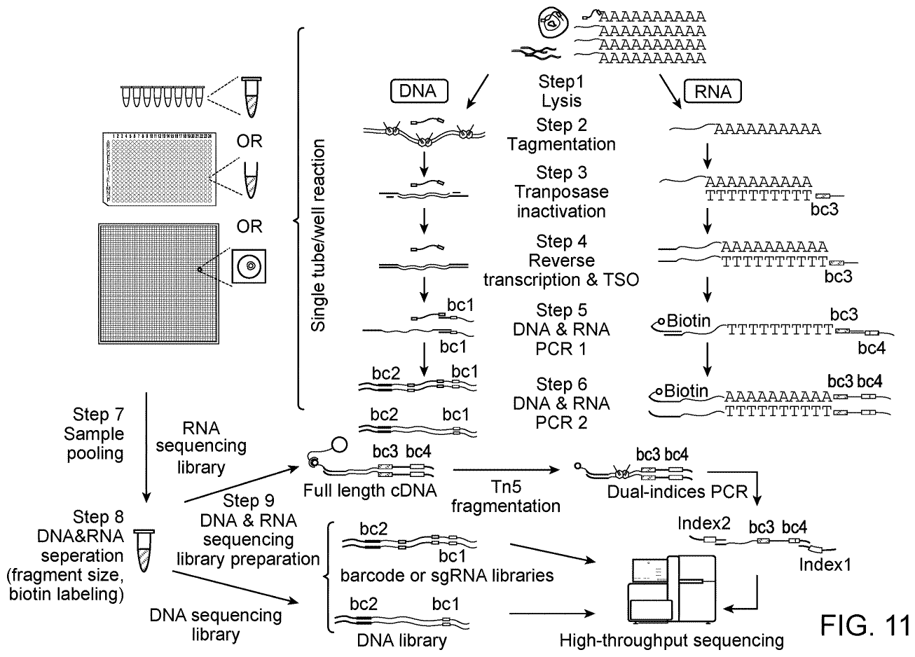

In some aspects, the amplified DNA can be used for profiling copy number variations/alterations (CNV/CNA), structure variations (SVs), indels and point mutations. In some aspects, the amplified DNA can be used for profiling targeted genes or gene panels, probe-based target capture, exon capture or other capture applications FIG. 9. In some aspects, the amplified DNA can be used for investigating DNA rearrangements and markers, detecting different frequency of mutations, profiling epigenetics modifications, DNA and protein interactions, and other DNA related applications.

In some aspects, the amplified RNA product can be from mRNA, small RNA, non-coding RNA, ribosome RNA, or combinations thereof, using different input materials. In some aspects, the amplified RNA product can be full length RNA, or fragmented RNA. In some aspects, the amplified RNA can be used to assemble transcriptomes, quantify gene expression, perform differential gene expression analysis and allele specific gene expression analysis, identify alternative gene-splicing events, study gene regulatory networks, infer gene expression (RNA) trajectories and velocities, and discover miRNAs, or other small noncoding RNAs and their differential expression.

In some aspects, the sequencing comprises paired-end sequencing or single-read sequencing.

In some aspects, the sequencing comprises next-generation sequencing (NGS).

The amplified DNA or cDNA library can be sequenced and analyzed using methods known to those of skill in the art, e.g., by next-generation sequencing (NGS). In certain exemplary aspects, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLID, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

In some aspects, the method further comprises identifying a mutation in the DNA or RNA.

In some aspects, the expression profiling methods described herein are also useful for ascertaining the effect of the expression of one or more nucleic acid sequences (e.g., genes, mRNAs and the like) on the expression of other nucleic acid sequences (e.g., genes, mRNAs and the like) in the same cell or in different cells. This provides, for example, for a selection of alternate molecular targets for therapeutic intervention if the ultimate or downstream target cannot be regulated.

The expression profiling methods described herein are also useful for ascertaining differential expression patterns of one or more nucleic acid sequences (e.g., genes, mRNAs and the like) in normal and abnormal cells. This provides a battery of nucleic acid sequences (e.g., genes, mRNAs and the like) that could serve as a molecular target for diagnosis or therapeutic intervention.

In some aspects, wherein the mutation is an insertion, a deletion, or a substitution.

In some aspects, the mutation is a single nucleotide variation.

In some aspects, the mutation is associated with a phenotype of interest.

In some aspects, the method further comprises detecting genomic copy number variation.

In some aspects, the method further comprises performing transcriptome quantification or isoform analysis.

The advantages of the present methods are discussed throughout the disclosure.

It can be appreciated that in some aspects the disclosed methods, e.g., the wellDR-seq method, can be used for low-, middle- and high throughput whole genome (DNA) and transcriptome (RNA) co-amplification and library preparation. In some aspects, the disclosed methods use two different sets of adaptors to barcode DNA and RNA separately (one set for DNA, one set for RNA) from the same single cells or input materials. In some aspects, the approach uses different barcodes combinations to assign cell barcodes for each modality (DNA or RNA) of each cell, and then pools all of the barcoded amplified product together to prepare DNA and RNA sequencing libraries separately. In some aspects, the disclosed method is a flexible whole genome and transcriptome co-amplification method, which can add different adaptor sets to label and amplify DNA and RNA from the same input materials/single cell simultaneously.

In some aspects, the disclosed methods (e.g., wellDR-seq) assign sample/cell barcodes to DNA and RNA modalities from different input materials/single cells by a nested multiplexing PCR to index DNA & RNA simultaneously using different barcode combinations.

In some aspects, the disclosed methods (e.g., wellDR-seq) can amplify whole genomes and whole transcriptomes simultaneously from single cells, or tens to millions of cells, or alternatively from from extracted or unextracted DNA/RNA materials or limited materials.

In some aspects, the disclosed methods (e.g., wellDR-seq) enables preparing DNA sequencing libraries from the input materials/cells together after pooling, and also enables preparing the RNA sequencing libraries from all input materials/cells together after pooling, which avoids preparing DNA and/or RNA sequencing libraries individually from each cell one-by-one. In some aspects, this feature allows for the claimed methods (e.g., wellDR-seq) to be highly scalable for both tubes or plates formats, as well as very high-throughput platforms such as nanowells or nanochips.

Certain aspects of the disclosure comprise the addition of adaptors to DNA fragments and RNA/cDNA in a mixed solution of the nucleic acids and does not require physical separation of the DNA and RNA nucleic acids during addition of the adaptors. This is in contrast to other methods of creating genomic and transcriptomic libraries from a single source like G&T-seq (Macaulay et al., Nat Methods 12, 519-522 (2015)), SIDR-seq (Han et al., SIDR: Genome Research 28, 75-87 (2018)), and DNTR-seq (Zachariadis et al., Molecular Cell 80,, 541-553.e5 (2020)). Further, the certain methods disclosed herein have other advantages over DR-Seq including that the present methods can be used for high-throughput analysis. For example, the method of DR-Seq first entails reverse transcription, then uses quasilinear amplification to amplify DNA and RNA. Because of this quasilinear amplification strategy, DR-Seq cannot achieve high throughput cell barcoding. Further, the DNA and RNA library of each single cell needs to be prepared separately in DR-Seq, which requires effort and cost to prepare (Dey, et al., Nat Biotechnol 33, 285-289 (2015)).

Cretan aspects of the present disclosure, e.g., the methods (e.g., wellDR-seq) are different from the scONE-seq method (Wu, et al., (2021)). For example, scONE-seq uses the same adaptors to label DNA and RNA, which does not allow the separation of DNA and RNA molecules during library preparation. This also does not allow users to control the sequencing depth for the DNA and RNA assays, which need to be sequenced at different depths. In contrast, certain methods disclosed herein use different adaptor combinations to distinctly label DNA and RNA. In some aspects, the assays (DNA or RNA) can then be enriched during the pre-amplification step, post-amplification step, and also after merging all of the libraries from all cells. In addition to different adaptors, in some aspects in the methods disclosed herein, the fragment size of DNA and RNA assay can also distinguish DNA and RNA. In some aspects, the methods of the disclosure (e.g., wellDR-seq) comprise labelling the adaptors or primers of either assay (DNA or RNA) with base modifications (eg. biotin) to further separate DNA and RNA assays after co-amplification.

In some aspects, certain methods of the present disclosure (e.g., wellDR-seq) uses combinatorial barcodes for both DNA and RNA assays, which can enable profiling the genome and transcriptome from hundreds or thousands of cells or low input materials. In some aspects, the methods disclosed herein (e.g., wellDR-seq) is flexible and can enrich either DNA or RNA pool, to prepare different libraries for a variety of sequencing purposes. The methods provide for alternative ways to label DNA and RNA, which allows sequencing full length RNA, 3′ RNA, 5′ RNA at large scales.

In some aspects, the present methods use the substrate specificity of the transposase and RNA ligase enzymes to selectively attach DNA-specific adapters to DNA and RNA-specific adapters to RNA, respectively in the pooled mixtures.

### VII. Kits

Certain aspects of the present disclosure are directed to a kit for performing the method of claim 1 comprising: a) a Class 2 transposase; b) a transposable oligonucleotide comprising an oligonucleotide adapter comprising a common priming site for DNA-specific amplification; c) a 5′ oligonucleotide adapter comprising a 5′ common priming site for RNA-specific amplification; d) a 3′ oligonucleotide adapter comprising a 3′ common priming site for RNA-specific amplification; k) an RNase inhibitor; l) a reverse transcriptase; m) a DNA polymerase; n) a set of DNA indexing PCR primers; and o) a set of RNA indexing PCR primers.

In some aspects, the kit further comprises reagents for performing next-generation sequencing.

The above-described reagents, including a Class 2 transposase (e.g., hyperactive Tn5 transposase), oligonucleotide adapters (e.g., adapter comprising a common priming site for DNA-specific amplification, a 5′ adapter comprising a 5′ common priming site for RNA-specific amplification, a 3′ oligonucleotide adapter comprising a 3′ common priming site for RNA-specific amplification), RNase inhibitor, reverse transcriptase, DNA polymerase (e.g., Taq polymerase for PCR), DNA indexing PCR primers; and RNA indexing PCR primers can be provided in kits with suitable instructions and other necessary reagents in order to carry out preparation of RNA and DNA sequencing libraries as described above. In some aspects, the kit will contain in separate containers the various primers, adapters, and enzymes, and other reagents required to carry out the method. In some aspects, instructions (e.g., written, CD-ROM, DVD, flash drive, SD card, digital download etc.) for preparing RNA and DNA sequencing libraries simultaneously as described herein will be included with the kit. The kit may also contain other packaged reagents and materials (e.g., wash buffers, nucleotides, silica spin columns, capture probes for ribosomal RNA depletion, and other reagents and/or devices for performing e.g., clonal amplification, digital PCR, NGS sequencing, ribosomal RNA depletion, nucleic acid purification, and the like).

All of the references cited above, as well as all references cited herein, are incorporated herein by reference in their entireties.

Any examples provided herein are offered by way of illustration and not by way of limitation.

### Examples

### Example 1: Well-DR-seq Method

### Overview of Well-DR-Seq Method

A method for co-amplification that can be used to generate DNA and RNA libraries for analysis, e.g., sequencing was developed. The “wellDR-seq” refers to a method that can barcode DNA and RNA materials independently, and amplify the barcoded materials simultaneously without physical separation of the DNA and RNA from single cells or low input materials before pooling all of the barcoded libraries together.

An exemplary workflow is show in FIG. 1. The barcoded DNA and RNA libraries are separated after pooling of all of the barcode libraries from all of the cells to further construct the DNA and RNA sequencing libraries individually, before loading to sequencers for high throughput sequencing. wellDR-seq involves several major steps including: (1) cell lysis, to break the cell and nuclear membrane and remove chromatin (2) Adaptor attachment to DNA, to add adaptors to DNA to distinguish between DNA and RNA libraries after pooling, such as using tagmentation reactions (e.g., Tn5 transposome) to fragment DNA into small fragments and add the adaptors at the same time. (3) Adapter attachment to RNA, to add adaptors (different from DNA specific adaptors) to RNA/cDNA molecules during reverse transcription and/or second strand synthesis, such as using polyT tailed primers with universal sequences (RNA adaptor) and template switching oligonucleotides (TSO). (4) DNA and RNA amplification, to amplify both DNA and RNA through the aforementioned DNA and RNA specific adaptors and to add the cell barcode to DNA and RNA in each tube or well at the same time. Different adaptors are used to label DNA and RNA, therefore wellDR-seq can enrich one of the modalities (eg. RNA) as desired before or after exponential co-amplification, especially when one modality amount is much less than the other one. (5) Sample pooling, to pool all of the libraries (barcoded DNA and RNA molecules) from each tube/well together. (6) DNA&RNA separation, to separate DNA and RNA libraries based on molecular features (eg. fragment size, biotin labels or different adapter sequences) (7) preparation of DNA and RNA sequencing libraries, which involves preparing DNA sequencing libraries for both barcoded DNA and barcoded RNA libraries independently. (8) Sequencing and data analysis, which involves computationally matching the data from DNA and RNA data, and perform more detailed analysis.

### Input Materials

Input material for wellDR-seq can be single cell/nucleus or low input materials, for example: several cells/nuclei and multiple cells/nuclei, can be processed, modified, fixed, tagmented or antibody attached cells/nuclei, can be organoids, a sample (e.g., small chunk/piece) of tissue(s), methanol-fixed or formalin-fixed, paraffin-embedded (FFPE) tissue samples, blood drops, buffy coat, body fluids, swabs, naked DNA/RNA etc. To achieve the preparation DNA and RNA libraries simultaneously, the first step of this method is to release DNA and RNA from input material into the reaction. The release procedure can use an enzyme-based method (e.g., protease), chemical-based methods (eg. detergents such as tween-20, Triton X-100), mechanical, acoustic, electrical based methods, or the combination of those methods. This step breaks the cellular/nuclear membrane and digests the chromatin structures to expose the DNA and RNA. RNase inhibitors may be added at this step to further prevent RNA from degradation during lysis step.

### DNA Adaptor Addition

To label the DNA molecules, the lysate can be used to perform reactions such as tagmentation using transposome (eg. Tn5 transposome). The transposome can be commercial and loaded such as the Tn5 transposome (Illumina) with universal oligonucleotides, or the transposome can be assembled by combining the transposase with transposase recognized DNA oligonucleotides (eg. Mosaic End (ME) sequences). The oligonucleotides that attach to the transposase can have the barcode or part of the barcode sequence on it for distinguishing different cells, while the oligonucleotides also serve as the identifier to distinguish DNA and RNA molecular in the same single cell/nuclei or input material. The transposome is then inactivated or inhibited by protein denaturing detergents (eg. SDS) or heating with EDTA or other inhibitors after the tagmentation reaction. The lysate can be used to perform random priming using the adaptor (primer) sequences with random sequences to generate short DNA fragments, the sequence should contain part of universal sequence severing as DNA adaptors.

### RNA Adaptors Addition

To profile the transcriptomics of single cell/nucleus or low input materials, the RNA or cDNA molecules are attached to the RNA modality with specific adaptors. For example, the RNA molecules are first converted into cDNA (complimentary DNA) by reverse transcriptase, including but not limited to MMLV reverse transcriptase, AMV reverse transcriptase or other RT enzymes. The primer used for reverse transcription (RT) can prime using RNA molecules with poly-T tails, or random sequences, as well as other targeted gene specific sequences. The primer also has a universal sequence component (>=6 bp) that is used as the RNA adaptor (PCR amplification handle) sequence for amplifying the cDNA in the later steps. The primer can also have a cell barcode or part of a cell barcode serving as cell identity. The primer can also have a unique molecular identifier (UMI) sequence to distinguish individual transcripts from PCR duplicate reads. The primer can also have modifications (such as biotin) used to separate the RNA from DNA molecular afterwards. The cell barcode can be composed of 1 or 2 oligonucleotide sequences, or multiple oligonucleotide sequences. During the RT reaction, the template switch oligonucleotides (TSO) can be added to form the second end of the cDNA sequences (as with the other RNA adaptor). The TSO sequences can have a cell barcode or part of a cell barcode, serving as a cell identity, and can also have a unique molecular identifier (UMI) sequence to distinguish individual transcripts from PCR duplicate reads. TSO can also have modifications (such as biotin) used to separate the RNA from DNA molecules afterwards. During lysis or other steps, the RNA molecule can become fragmented or lose the 5′ cap structure, to facilitate TSO procedure, the RNA can be treated with a capping reagent (eg Vaccinia Capping System of New England BioLabs) before performing the RT step. Alternatively, instead of using the TSO strategies, other methods can be used to add the RNA adaptor (PCR priming) sequences to the second end of the cDNA molecules, such as performing a ligation reaction at the 3′end of the cDNA, or performing a second strand synthesis using oligonucleotides with random priming sequences and universal tails, or oligonucleotides with target priming sequence and universal tails. The RNA adaptors (PCR priming) sequences could have modifications (such as biotin) that will be used to separate the RNA from DNA molecules after the initial amplification reactions. To facilitate the separation of DNA and RNA assay after pooling all of the samples (or cells) together and prepare the DNA and RNA libraries separately, the two sequences used to label the DNA (eg, oligonucleotide attached on ME sequence of the transposome) and the two sequences used to label the RNA (eg, RT primers, TSO sequences) will have at least two distinct sequences.

### Cell/Material Barcode Addition for DNA and RNA Modalities and DNA and RNA Coamplification Reactions

WellDR-seq uses different adaptor s to label DNA and RNA separately, which makes pre-amplification of one of the individual assays possible. To enrich the number of molecules from one of the assays (eg. RNA assay), both of the forward and reverse primers of the assay that needs to be enriched can be added, while only adding one of the primers of the other assay. Or add one or two primers (forward or/and reverse) to the assay that need to be enriched, but then do not add any primers for the other assay. The one assay enrichment procedure can take place before the DN and RNA exponential co-amplification (pre-enrichment) or afterwards (post-enrichment). The primers can have a cell barcode or part of a cell barcode serving as a cell identity, the primers can also have modifications (such as biotin) that are used to separate the pool of RNA from DNA molecules afterward. The primer pairs for the DNA assay and pairs for the RNA assay are all added into the same reaction during exponential co-amplification. The primers can have cell barcodes or part of cell barcodes serving as the cell identity, and the primers can also have modifications (such as biotin) used to separate the RNA from DNA molecules afterwards. The annealing temperatures of the co-amplification step can be used to favor one of the assays or both assays to control the total number of molecules amplified. By controlling the favored annealing temperature, the DNA and RNA assay product amounts can be balanced in alternative manner.

### Cell/Low-Input Material Barcoding of DNA and RNA Individually

Before pooling the DNA and RNA fragments from all of the cells/sample together, the DNA or RNA from the same cell/sample are labelled with cell/sample barcodes. The DNA and RNA from the same cell/sample material can be labelled with same barcode sets or different barcode sets (by knowing the DNA/RNA barcode correspondence relationship). The DNA and RNA modalities can be barcoded by single or multiple barcodes, or the combination of barcodes. The two barcodes, three barcodes or multiple barcodes (>3) can be located in one end (eg 5′) of each modality, or alternatively can be located in both ends (eg 5′ and 3′) of each modality. The wellDR-seq method can use tagmentation based chemistries (eg. Tn5 transposome) to fragment DNA. The DNA fragments can be barcoded by using tagmentation enzyme with different adaptors (eg. by attaching different oligonucleotide sequences to the mosaic sequences of Tn5 transposase), or can be barcoded in the subsequent PCR steps through PCR primers that contain different barcodes (indices) or barcode combinations, or barcoded by both approaches by combining the barcode introduced in tagmentation step and PCR steps. Furthermore, the barcoding for the RNA assay, can occur at the reverse transcription (RT) step by using different RT primers, or barcoded at the later PCR steps through different PCR primers with different barcodes (indices) or barcode combinations, which can be barcoded by combining the barcode introduced in the reverse transcription step and PCR steps. In order to separate the DNA and RNA libraries after pooling all of the libraries from single cells or sample materials, DNA and RNA libraries in each single cell or low input sample are labeled with different adaptors, which will be used to distinguish these two different libraries after pooling. Importantly, at least two of these adaptors are different between the DNA and RNA libraries to ensure the procedure of preparing DNA and RNA libraries separately can occur after pooling. One of the adaptors or primers from the DNA or RNA assay may have specific one or multiple modifications (such as biotin) to help separate the two assays after pooling.

### Sample Pooling and DNA&RNA Separation

After molecules from DNA and RNA assays of the same cell/material are labelled with cell barcodes, the libraries from all of the reaction wells are pooled together. From this pool of cells/samples, a physical separation of the amplified DNA and RNA libraries was performed. The separating methods can be based on the DNA/RNA fragment sizes, can be based on the modifications that are used to label one of the modalities (eg. Biotin modifications), can be based on different adaptors used to distinguish the DNA and RNA modalities, or can be other methods that can distinguish amplified DNA and RNA molecules.

### DNA and RNA Sequencing Library Construction

After the separation of the DNA and RNA modalities is completed, the DNA and/or RNA can be used for high-throughput sequencing platforms if the PCR primers used to amplify the DNA modality are the same sequencing adaptor that is used for sequencing. For example, if the Illumina Tn5 transposome (eg TDE1) is used during the tagmentation step, and the PCR primers used to amplify DNA modality are Nextera PCR forward and reverse primers, then after separation of the DNA and RNA, the DNA library is ready-to-load for sequencing libraries. The separated DNA and/or RNA amplification products can be used to prepare different sequencing libraries according to the research purpose and sequencing instrument requirements. The separated DNA and/or RNA amplification products can also be used for further enrichment using DNA or RNA specific adaptors added during the previous steps. Further enriched products can be used as input materials for performing high throughput sequencing platforms if the PCR primers used to amplify the DNA modality are the same sequencing adaptor used for the sequencing reactions. These methods can be modified to prepare different sequencing libraries according to the research purpose and sequencing instrument requirements. High throughput sequencing platforms include, but are not limited, to next generation sequencing, single molecule sequencing and nanopore sequencing.

The amplified DNA can be used for profiling copy number variations/alterations (CNV/CNA), structure variations (SVs), indels and point mutations. They can also be used for profiling targeted genes or gene panels, probe-based target capture, exon capture or other capture applications. They can also be used for investigating DNA rearrangements and markers, detecting different frequency of mutations, profiling epigenetics modifications, DNA and protein interactions, and other DNA related applications.

The amplified RNA product can be from mRNA, small RNA, non-coding RNA, ribosome RNA, or combinations thereof, using different input materials. The amplified RNA product can be fulllength RNA, or fragmented RNA. The amplified RNA can be used to assemble transcriptomes, quantify gene expression, perform differential gene expression analysis and allele specific gene expression analysis, identify alternative gene-splicing events, study gene regulatory networks, infer gene expression (RNA) trajectories and velocities, and discover miRNAs, or other small noncoding RNAs and their differential expression.

### Example 2: Performing wellDR-Seq of Single Cells in Single Tubes/96-Well Plates

The wellDR-seq method was tested using single cells in single tubes and 96-well plates. First, 2 ul lysis buffer mix containing 0.37×PBS, 2.5% Tween-20, 0.25% TritonX-100, 15 mM Tris-HCL, pH 8.0, 1×2nd diluent (Takara), 0.75 U/ul RNase inhibitor, and 1.07 mAU/ul protease (Qiagen) were added into each tube or well. Then single cells were sorted individually into each tube/well using Melody (BD Bioscience) (1 cell/tube or 1 cell/well). Lysis was carried out at 55° C. for 10 min and protease was inactivated at 70° C. for 15 min. Next, 2 ul of tagmentation mix containing 1.92×TD buffer (Illumina), 0.6 U/ul RNase inhibitor, and 0.05 μl TDE1 (Illumina) were added to each tube/well. Tagmentation reaction was carried out at 55° C. for 5 min. Afterwards, 4 μl of neutralization mix containing 18.75 mM EDTA, 41.25 mM DTT, 5 mM dNTPs, 0.75 U/ul RNase inhibitor, and 2.5 μM RNA_S5XX primers

[GAGGCGTAGTGGCTTAGATCGCTTTTTTTTTTTTTTTTTT
TTTTTTTTTTTTVN (SEQ ID NO: 1)]

were added to each tube/well. Neutralization was carried out at 50° C. for 15 min and 72° C. for 3 min. 4 ul of RT mix containing 3× SuperScript IV RT buffer (Invitrogen), 33.75 mM MgCl2, 0.7 U/ul RNase inhibitor, 2.75 mM dNTPs, 8.75 U/ul SuperScript IV (Invitrogen), and 3 μM 5′ biotin modified TSO primers [/5Biosg/TCTCCGACTCAGTACATrGrGrG (SEQ ID NO: 2)] was directly added into each tube/well. Reverse transcription was carried out at 50° C. for 60 min, and the reaction was stopped by incubating at 80° C. for 10 min. Lastly, 38 μl of PCR mix containing 1.3×KAPA HiFi HotStart Ready Mix (Roche), 0.92 μM of 5′ biotin modified TSO primers, 0.92 μM of RNA_N7XX primers [AAGCAGTGGTATCAACGCAGAGTAC-N8 (8 bp, RNA_Cell_Barcode1)-NNNNNNNNNNGAGGCGTAGTGGCT (SEQ ID NO: 3)], 0.4 uM of DNA_N7XX primers [CAAGCAGAAGACGGCATACGAGAT-N8 (8 bp, DNA Cell Barcode2)-GTCTCGTGGGCTCGG (SEQ ID NO: 4)], and 0.4 μM of DNA_S5XX primers [AATGATACGGCGACCACCGAGATCTACAC-N8 (8 bp, DNA_Cell_Barcode1)-TCGTCGGCAGCGTC (SEQ ID NO: 5)] were added into each well/tube. Co-amplification PCR of DNA and cDNA was cycled as follows: 7 cycles of 98° C. for 20 s, 55° C. for 30 s, 72° C. for 90 s, then 72° C. for 2 min.

### Example 3: Performing wellDR-Seq of Single Cells in 384-Well Plates

Single cells were FACS-sorted into 384-well plates using elody (BD) at 1 cell/well. All of the reagents were dispensed into 384-well plates using an acoustic liquid handler Echo525 (Labcyte). First, 200 nl lysis buffer mix containing 0.37×PBS, 2.5% Tween-20, 0.25% TritonX-100, 15 mM Tris-HCL, pH 8.0, 1×2nd diluent (Takara), 0.75 U/ul RNase inhibitor and 1.07 mAU/ul protease (Qiagen) were added to each well. Lysis was carried out at 55° C. for 10 min and protease was inactivated at 70° C. for 15 min. Next, 200 nl tagmentation mix containing 1.92×TD buffer (Illumina), 0.6 U/ul RNase inhibitor, and 5 nl TDE1 (Illumina) were added to each well.

Tagmentation reaction was carried out at 55° C. for 5 min. Afterwards, 400 nl of neutralization mix containing 18.75 mM EDTA, 0.7× SuperScriptIV RT buffer (Invitrogen), 7.8 mM dNTPs, 0.75 U/ul RNase inhibitor, and 2.5 μM RNA_S5XX primers were added to each well. Neutralization was carried out at 50° C. for 15 min a 72° C. for 3 min. 400 nl of RT mix containing 2.3× Superscript IV buffer (Invitrogen), 41.25 mM DTT, 33.75 mM MgCl2, 0.7 U/ul RNase inhibitor, 8.75 U/ul SuperScript IV (Invitrogen), and 3 μM TSO primers was directly added into each well. Reverse transcription was carried out at 50° C. for 60 min, and the reaction was stopped by incubating at 80° C. for 10 min. cDNA preamplification was immediately performed by adding 3.75 μl cDNA pre-amplification mix containing 1.6×KAPA HiFi HotStart Ready Mix (Roche), 1.33 μM TSO primers, 1.33 μM RNA_N7XX primers, and 1.33 μM DNA_N7XX primers were added into each well. cDNA pre-amplification PCR was cycled as follows: 72° C. for 3 min, 98° C. for 3 min, 8 cycles of 98° C. for 20 s, 55° C. for 30 s, 72° C. for 90 s, then incubated at 72° C. for 5 min. Lastly, 1.33 μM of DNA_S5XX primers were added into each well. Co-amplification PCR of DNA and cDNA was cycled as follows: 98° C. for 3 min, 10 cycles of 98° C. for 20 s, 55° C. for 30 s, 72° C. for 90 s, then incubated at 72° C. for 5 min.

### Example 4: Well-DR Nano-Well Assay

Single cell suspensions were stained with ReadyProbes Cell Viability Imaging Kit, Blue/Red (Thermo) at 37° C. for 15 min. Cells were spun down at 400 g for 5 min at 4° C. and resuspended in 1×PBS. The single cell suspensions were further diluted to 32,000 cells/ml with resuspension buffer containing 1×PBS, 1×2nd Diluent (Takara), and 1.2 U/ul RNase inhibitor. Next, diluted cell suspension were dispensed into a 350 nl nanowell chip (Takara) using the ICELL8 CX system (Takara). The chip was scanned and only nanowells containing viable singlets were selected for downstream experiments. Then, 35 nl lysis buffer mix containing 7.6% Tween-20, 0.76% TritonX-100, 45.75 mM Tris-HCL, pH 8.0, 1.5 U/ul RNase inhibitor and 2.14 mAU/ul protease (Qiagen) were added into each well. Lysis was carried out at 55° C. for 20 min and protease was inactivated at 70° C. for 15 min. Next, 35 nl tagmentation mix containing 1.62×TD buffer (Illumina), 0.6 U/ul RNase inhibitor, and 6.125 nl TDE1 (Illumina) were added to each well. Tagmentation reaction was carried out at 55° C. for 12 min. Afterwards, 35 nl of neutralization mix containing 37.6 mM EDTA (Invitrogen), 0.57× SuperScriptIV RT buffer (Invitrogen), 3.9 mM dNTPs, 3.9 mM additional dCTPs, 1.5 U/ul RNase inhibitor, and 2.5 μM RNA_S5XX primers were added to each well. Neutralization was carried out at 50° C. for 30 min and 72° C. for 3 min. 50 nl RT mix containing 3.4× Superscript IV buffer (Invitrogen), 52.25 mM DTT (Invitrogen), 42.9 mM MgCl2, 1.1 U/ul RNase inhibitor, 15.8 U/ul SuperScript IV (Invitrogen), and 3 μM TSO primers was directly added into each well. Reverse transcription was carried out at 50° C. for 60 min, and the reaction was stopped by incubating at 80° C. for 10 min. cDNA preamplification was immediately performed by adding 35 nl DNA_N7XX and RNA_N7XX mix containing 1×KAPA high GC buffer (Roche), 5 μM DNA_N7XX primers and 7.5 μM RNA_N7XX primers and 35 nl KAPA enzyme mix containing 1×KAPA high GC buffer (Roche), 9.4 μM TSO primers, and 0.17 U/ul KAPA HiFi HotStart Polymerase (Roche). cDNA pre-amplification PCR was cycled as follows: 72° C. for 10 min, 98° C. for 3 min, 6 cycles of 98° C. for 20 s, 55° C. for 30 s, 72° C. for 150 s. Final elongation was performed for 5 min at 72° C. Finally, 35 nl DNA_S5XX primers mix containing 5 μM DNA_S5XX primers and 1×KAPA high GC buffer (Roche) were added into each well. Co-amplification PCR of DNA and cDNA was cycled as follows: 98° C. for 3 min, 10 cycles of 98° C. for 20 s, 60° C. for 30 s, 72° C. for 90 s. Final elongation was performed for 5 min at 72° C.

### Example 5: Well-DR DNA and cDNA Library Preparation

After the DNA and cDNA co-amplification was completed, the amplified products were pooled into one tube. The pooled sample was first double-selected by 0.6×-1.8× Ampure beads (Beckman) purification to separate DNA final library and full-length cDNA. The purified full-length cDNA was further purified by 0.8× Ampure beads purification. Next, the full-length cDNA was captured by M270 beads (Invitrogen) and PCR mix containing 0.05Uul KAPA HiFi HotStart DNA Polymerase (Roche), 0.3 μM DDR_PCR_P5 primers (AATGATACGGCGACCACCGAGATCTACACGCCTGTCCGCGGAAGCAGTGGTATCA ACGCAGAGTA C (SEQ ID NO: 6)) and 0.3 μM 5′ biotin modified TSO primers [/5Biosg/TCTCCGACTCAGTACATrGrGrG (SEQ ID NO: 7)] was added to perform PCR on beads. The PCR was cycled as follow: 98° C. for 3 min, 16-24 cycles of 98° C. for 20 s, 69° C. for 30 s, 72° C. for 150 s. Final elongation was performed for 5 min at 72° C. The amplified full-length cDNA was purified by two times of 0.6× Ampure beads purification. cDNA library was prepared by using Ing of purified full-length cDNA with 5 ul ATM enzyme (Illumina) and 1×TD buffer (Illumina). The tagmentation reaction was performed at 55° C. for 5 min. Then 5 μl of NT buffer (Illumina) was immediately added and neutralization reaction was performed by incubating at room temperature for 5 minutes. Afterwards, the cDNA lib amplification was carried out by adding 0.3 μM DDR_PCR_P5 and standard Illumina P7 adaptors

[CAAGCAGAAGACGGCATACGAGAT-N8(8bp)-
GTCTCGTGGGCTCGG (SEQ ID NO: 8)]

and 15 μl NPM. The CR was cycled as follow: 95° C. for 30 s, 98° C. for 3 min, 12 cycles of 98° C. for 10 s, 55° C. for 30 s, 72° C. for 30 s. Final elongation was performed and A library was selected by 0.6× Ampure beads purification.

### Example 6: Low-Throughput Single Tube wellDR Experiments Using SK-BR-3

A single tube test experiment was performed following the protocol described in Example 2. In total, the copy number aberrations (DNA) and gene expression (mRNA) of 12 single cells from the SK-BR-3 breast cancer cell line were profiled. For the DNA libraries, 13.6 M reads were sequenced in total (1.13 M per cell in average) with a mean PCR duplicates rate of 5.25%, 63 reads per bin at 220 kb resolution were obtained.

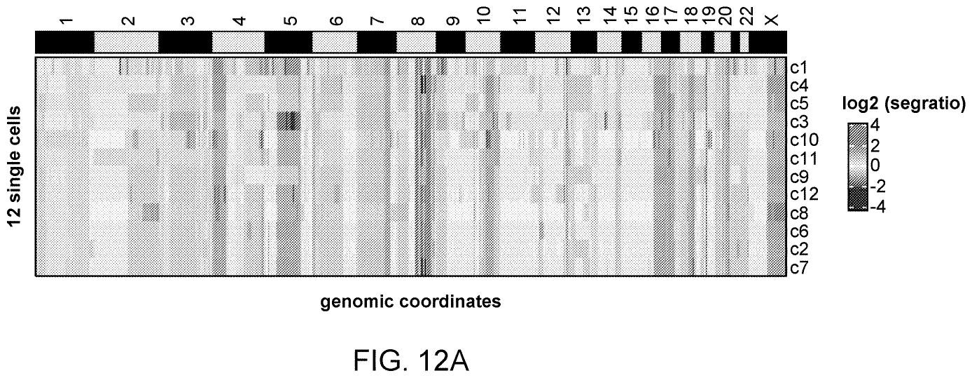

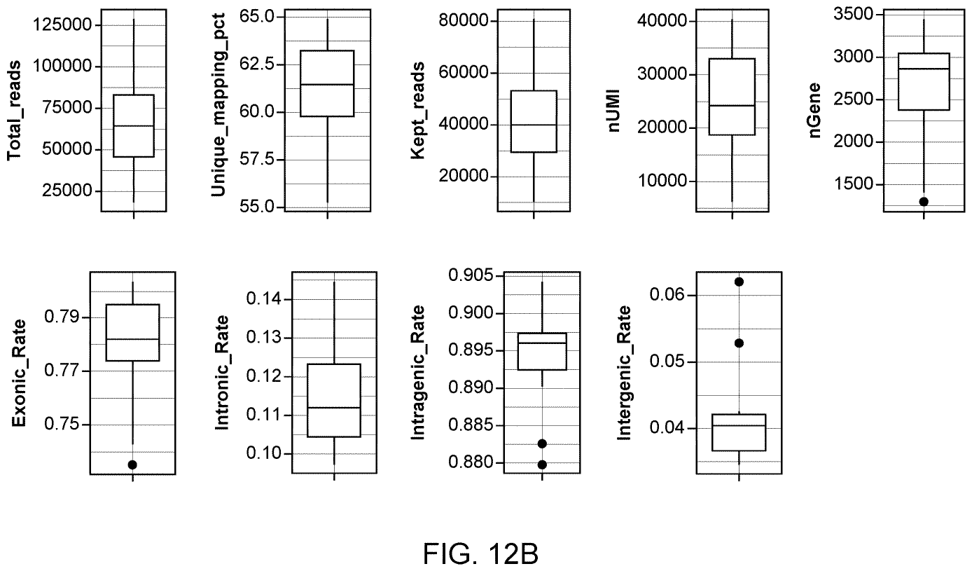

Next, heatmaps for the 12 single cells were constructed using log 2 segment ratios (FIG. 12A), wellDR-seq correctly identified the common copy number gains including chr1q, chr7, chr8q (MYC), chr17 (focal amplification, ERBB2) and chr20q, as well as copy number losses including chr4p, chr10q, chr17p, that have been reported in other studies (eg. Navin et al. 2011, Nature). In terms of the RNA data, 65,133 reads per cell on average were sequenced with a unique mapping rate of 61.28%. On average, 40,337 reads per cell were retained for the downstream data analysis. In the 12 single cells, wellDR detected 23,607 unique molecular identifier (UMI) and 2,648 genes per cell in average in the RNA data. Based on mapping, it was found that 77.88% reads were successfully mapped to the exonic region, while only 4.19% of reads mapped to the intergenic region, showing high QC metrics for the data (FIG. 12B).

### Example 7: Mid-Throughput wellDR Experiments in MDA-MB-231 Using 384 Well Plates

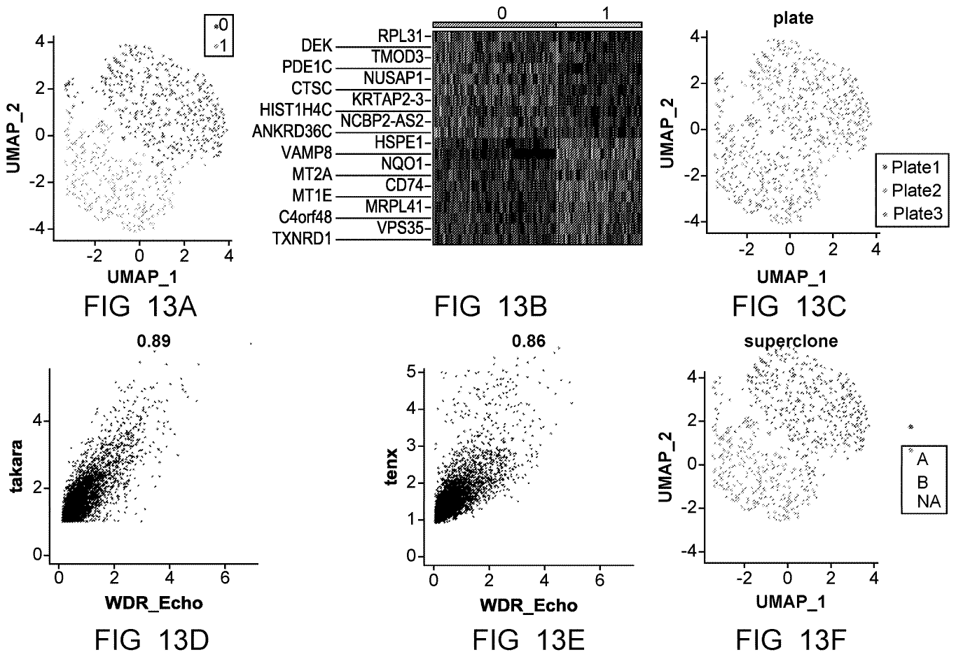

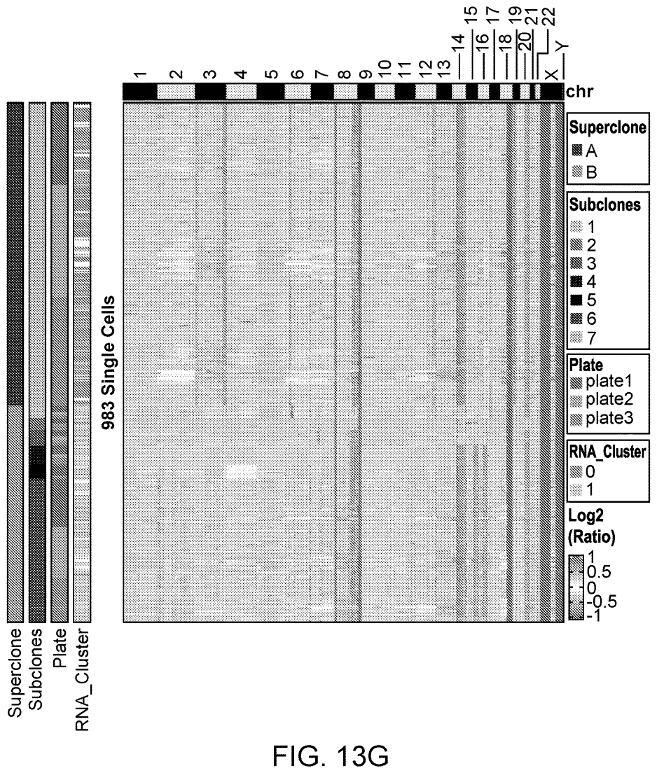

According to the protocol in Example 3, wellDR-seq was used to profile the genome and transcriptome of single cells from the MDA-MB-231 cell line using 384-well plates. The wellDR-seq libraries were prepaid from three 384-well plates. In each plate, single cells were sorted into 380 wells, 10 cells to two wells as positive control and zero cell to the other two wells as negative controls. For the RNA libraries, 80 M reads were sequenced in total (20-50 k reads/cell), of which 62% mapped to the transcriptome regions. A total of 767 (67%) single cells passed QC. On average, 21,641 UMI and 3,317 genes were detected in each single cell. The 767 single cells clustered into two major clusters based on their gene expression profiles (FIGS. 13A-13B). The cells from the three different plates were well mixed in the clusters, suggesting minimal batch effects (FIG. 13C). The gene expression profiles measured by wellDR-seq is highly consistent with the profiles measured by ICELL8 3′DE (Takara, p=0.89) and 10× Genomics single cell 3′ RNA-seq data (p-0.86) of the same cell line (FIGS. 13D-13E). DNA copy number libraries were sequenced 939 k reads per cell on average with 12% PCR duplicate rates. On average, 824K reads were retained per cell after removing the PCR duplicates and other low-quality reads. There were 917 (80%) single cells that passed quality control. Next, 220 k variable bin resolution was used to infer copy number profiles of the single cells and clustered the DNA data based on the segmented copy number profiles. Two superclones and 7 different subclones were identified. The cells from different plates were well mixed in each superclone and subclone as well, indicating wellDR-seq is both accurate and reproducible (FIG. 13G). The two major RNA clusters matched well to the two DNA superclones in the heatmaps. Similarly, the two DNA superclones were mapped to the RNA UMAP, the DNA superclones and RNA clusters were well matched (FIG. 13F).

Taken together, this data suggests that wellDR-seq is not only able to distinguish tumor subclones at the RNA level, but also from the DNA profiles. Furthermore, by using the DNA data, wellDR-seq identified more clonal sub structure (e.g. subclones) while the gene expression programs of the same cells was very similar.

### Example 8: High-throughput wellDR Experiments in MDA-MB-231 using Nanowells

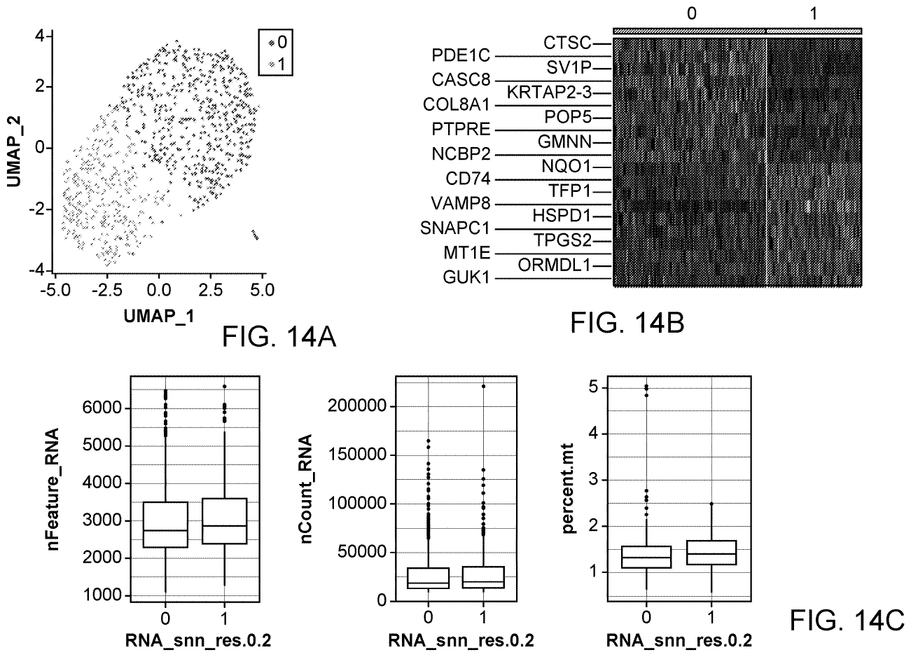

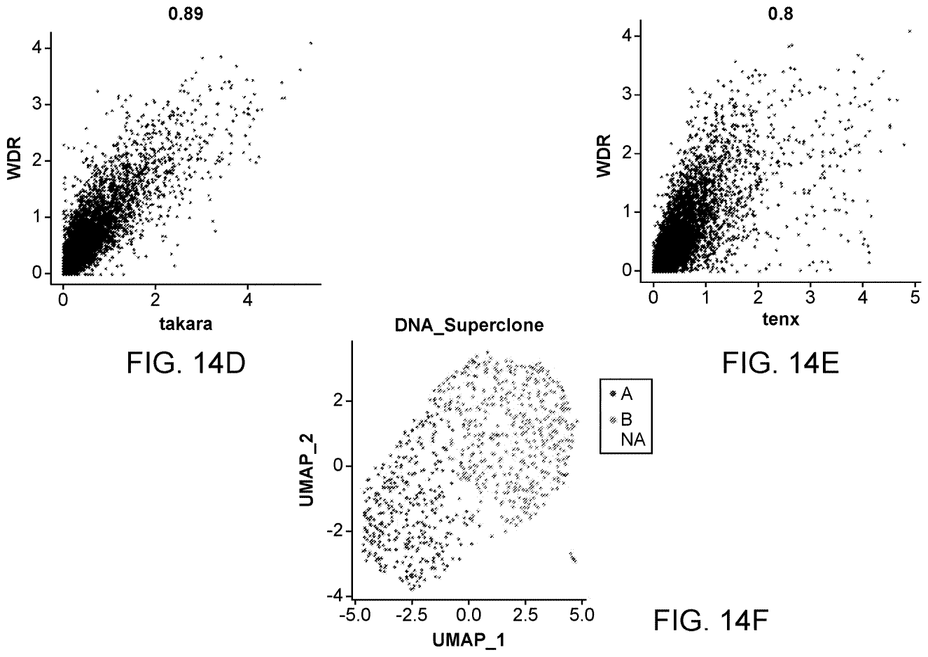

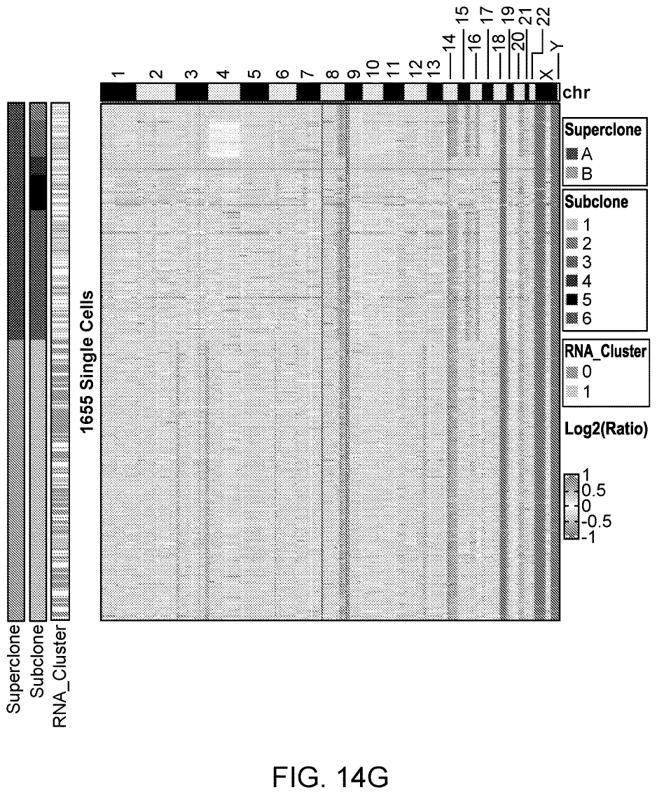

According to the protocol in Example 4, the wellDR-seq method was applied to amplify and prepare DNA and RNA libraries simultaneously from thousands of single cells in parallel from the MDA-MB-231 breast cancer cell line by using nanowell chips to demonstrate a high-throughput application. Single cell suspensions were dispensed into 5184-wells nanowell chips (ICELL8), and selected 1763 single cells to perform the wellDR-seq protocol. In the RNA data, 98 M reads were sequenced with the correct wellDR-seq RNA library structure, of which 80% of the reads mapped to the transcriptome. In total, 964 cells (55%) passed RNA QC with a median read of 34.98 k, median UMIs of 18.32 k and median gene average of 2817 per cell. Consistent with the results from the 384-well plates, all of the cells were clustered into two different clusters based on their gene expression profiles (FIG. 14B). This data showed similar gene counts, UMI counts, and mitochondrial percentages between the two major clusters (FIG. 14C). The detected gene expression profile is highly correlated with the results from ICELL8 3′DE (Takara, p=0.89) and 10× Genomics single cell 3′ RNA-seq data (p=0.8) using the same cell line as input materials (FIGS. 14D-14E). On the DNA side, 1655 (94%) single cells passed QC. On average, 577 k reads per cell were sequenced with 23% PCR duplicate rates. There were 32 reads (median) in each bin at 220K variable bin resolution. Based on the copy number aberration events, the single cells were clustered into two superclones and 6 subclones (FIG. 14G). Mapping the cells based on the superclones information into the UMAP space of RNA clustering, the two modalities (DNA and RNA) were found to be very well matched in high dimensional space (FIG. 14F). Inversely, mapping the RNA clusters to the DNA copy number profile heatmaps, showed that the RNA cluster also matched with DNA clustering results (FIG. 14G). These data suggest that the RNA expression programs reflect differences in the genotypes of the cancer subclones, validating the technical performance of the well-DR approach.

### Example 9: Performing wellDR-Seq in Single Tubes/96-Well Plates with RNA-First Labeling Chemistry

To perform wellDR-seq in single tubes/96-well plates with labeling RNA first chemistry, 4 ul lysis buffer mix containing 0.5×PBS, 2.5% Tween-20, 0.25% TritonX-100, 15 mM Tris-HCL, pH 8.0, 1×2nd diluent (Takara), 0.75 U/ul RNase inhibitor, 1.07 mAU/ul protease (Qiagen) and 1.5 μM RNA_S5XX primers

[GAGGCGTAGTGGCTTAGATCGCTTTTTTTTTTTTTTTTTT
TTTTTTTTTTTTVN (SEQ ID NO: 1)]

were added to each tube/well. Then single cells were sorted individually into each tube/well using Melody (BD Bioscience) (1 cell/tube or 1 cell/well). Lysis was carried out at 55° C. for 20 min and protease was inactivated at 70° C. for 15 min. Following lysis, the samples were incubated at 72° C. for 3 mins and added 2 ul RT mix into each tube/well on ice, which containing 3× SuperScript IV RT buffer (Invitrogen), 36 mM MgCl2, 0.7 U/ul RNase inhibitor, 5 mM dNTPs, 8.75 U/ul SuperScript IV (Invitrogen), and 3 μM 5′ biotin modified TSO primers

[/5Biosg/TCTCCGACTCAGTACATrGrGrG (SEQ ID NO: 2)].

Reverse transcription was carried out at 50° C. for 60 min, and the reaction was stopped by incubating at 80° C. for 10 min. Next, 6 ul of tagmentation mix containing 2×TD buffer (in-house), 20 mM MgCl2 and 0.2 μl TDE1 (Illumina) were added to each tube/well. Tagmentation reaction was carried out at 55° C. for 10 min. Afterwards, 2 μl of neutralization mix containing 210 mM EDTA were added to each tube/well. Neutralization was carried out at 50° C. for 30 mins. Lastly, two PCR programs were tested for the RNA first version of wellDR-seq in single tubes/well. The first PCR program is using 36 ul PCR mix buffer. 36 μl of PCR mix containing 1.38×KAPA HiFi HotStart Ready Mix (Roche), 0.82 μM of 5′ biotin modified TSO primers, 185 mM MgCl2, 0.46 μM of RNA_N7XX primers

[AAGCAGTGGTATCAACGCAGAGTAC-
N8(8 bp, RNA_Cell_Barcode1)-
NNNNNNNNNNGAGGCGTAGTGGCT (SEQ ID NO: 3)],
0.3 uM of DNA N7XX ;
[CAAGCAGAAGACGGCATACGAGAT-N8
(8 bp, DNA_Cell_Barcode2)-
GTCTCGTGGGCTCGG (SEQ ID NO: 4)],
and
0.3 uM of DNA_S5XX primers
[AATGATACGGCGACCACCGAGATCTACAC-
N8(8 bp, DNA_Cell_Barcode1)-
TCGTCGGCAGCGTC (SEQ ID NO: 5)]

primers
were added into each well/tube. Co-amplification PCR of DNA and cDNA was cycled as follows: 22 cycles of 98° C. for 20 s, 60° C. for 30 s, 72° C. for 90 s, then 72° C. for 5 min. The second PCR program is using 8 ul PCR program. Firstly, 6 ul PCR mix1 containing, 2.4×KAPA HiFi GC Buffer, 0.5 ul KAPA HiFi DNA Polymerase, 127 mM MgCl2, 6.6 mM dNTP, 0.36 μM of 5′ biotin modified TSO primers, 0.2 μM of RNA_N7XX primers

[AAGCAGTGGTATCAACGCAGAGTAC-N8
(8 bp, RNA_Cell_Barcode1)-NNN
NNNNNNNGAGGCGTAGTGGCT (SEQ ID NO: 3)].

Pre-amplification PCR of cDNA was cycled as follows: 8 cycles of 98° C. for 20 s, 60° C. for 30 s, 72° C. for 90 s were added into each well/tube, then 72° C. for 5 min. Next, 2 ul of PCR mix2 containing 0.5×KAPA HiFi GC Buffer, 0.13 μM of DNA N7XX primers

[CAAGCAGAAGACGGCATACGAGAT-N8(8 bp,
DNA_Cell_Barcode2)-
GTCTCGTGGGCTCGG (SEQ ID NO: 4)],
and
0.13 uM of DNA_S5XX_primers
[AATGATACGGCGACCACCGAGATCT
ACAC-N8(8 bp, DNA_Cell_Barcode1)-
TCGTCGGCAGCGTC (SEQ ID NO: 5)]

were added into each well/tube. Co-amplification PCR of DNA and cDNA was cycled as follows: 22 cycles of 98° C. for 20 s, 60° C. for 30 s, 72° C. for 90 s, then 72° C. for 5 min. The protocol as outlined in Example 5 was then followed to finish the final preparation of the DNA and RNA libraries.

What is claimed is:

1. A method of co-amplifying DNA and RNA from a single biological sample, the method comprising:
a) lysing a biological sample to release a plurality of nucleic acids comprising both genomic DNA (gDNA) and RNA from the biological sample;
b) fragmenting the gDNA in the plurality of nucleic acids;
c) attaching a DNA adaptor to the fragmented gDNA from (b) to form a plurality of DNA fragment-adaptor molecules;
d) synthesizing complementary DNA (cDNA) from the RNA in the plurality of nucleic acids, wherein the synthesizing comprises reverse transcription comprising a reverse transcriptase and an RNA primer, wherein the RNA primer comprises an RNA adaptor which is distinguishable from the DNA adaptor to form a plurality of cDNA-adaptor molecules; and
e) co-amplifying the plurality of DNA fragment-adapter molecules and the plurality of cDNA-adaptor molecules in the same reaction compartment, wherein one or more barcode sequences are added to the DNA-adaptor and cDNA-adaptor during amplification to form a plurality of DNA amplicons and a plurality of cDNA amplicons;
wherein (a)-(e) are performed in the same reaction compartment without physically separating the DNA from the RNA or cDNA.

2. The method of claim 1, wherein RNA is not fragmented during the fragmenting of the gDNA in (b).

3. The method of claim 1 or 2, wherein the synthesizing in (d) is performed concurrently with, before, or after (b) and/or (c).

4. The method of any one of claims 1-3, wherein the synthesizing in (d) is performed during, before, or after the lysing in (a).

5. The method of any one of claims 1-4, wherein the steps of (b-c) and (d) can occur concurrently.

6. The method of any one of claims 1-5, wherein the biological sample is selected from the group consisting of a plurality of cells, a single cell, an organoid, a tissue, a body fluid, naked nucleic acids, and any combination thereof.

7. The method of any one of claim 1-6, wherein the biological sample is a plurality of cells.

8. The method of any one of claims 1-6, wherein the biological sample is a single cell.

9. The method of any one of claims 6-7, wherein the single cell or the plurality of cells comprise a eukaryotic cell or a prokaryotic cell.

10. The method of any one of claims 1-9, wherein the biological sample comprises a genetically aberrant cell, cancer cell, or rare blood cell.

11. The method of any one of claims 6-10, wherein the cell or plurality of cells comprise a human cell.

12. The method of any one of claims 6-11, wherein the cell or plurality of cells comprise a live cell, a genetically engineered cell, a perturbed cell, or a fixed cell.

13. The method of any one of claims 6-12, wherein the cell or plurality of cells comprise a genetically engineered cell, an antibody attached to a single cell, a prelabelled single cell, or a barcoded single cell.

14. The method of any one of claims 1-14, wherein the biological sample comprises a micro-dissected tissue.

15. The method of claim 14, wherein the micro-dissected tissue is a fresh tissue.

16. The method of claim 14, wherein the micro-dissected tissue is a fixed tissue.

17. The method of any one of claims 1-16, wherein the biological sample is from a biopsy.

18. The method of any one of claims 1-17, wherein the biological sample is from a surgery sample.

19. The method of claim 6, wherein the body fluid is blood, urine, saliva, mucus, semen, vaginal fluid, amniotic fluid, cerebrospinal fluid, or a tissue fluid.

20. The method of any one of claims 1-19, wherein the lysing comprises enzymatic lysing, chemical lysing, mechanical lysing, acoustic lysing, electrical-based lysing, or any combination thereof.

21. The method of any one of claims 1-20, wherein the lysing in (a) further comprises adding an RNase inhibitor.

22. The method of any one of claims 1-21, wherein the attaching of the DNA adapter in (c) comprises tagmentation.

23. The method of claim 22, wherein the tagmentation comprises adding a Tn5 transposome.

24. The method of any one of claims 1-21, wherein the attaching of the DNA adapter in (c) comprises DNA ligation.

25. The method of any one of claims 1-21, wherein the attaching of the DNA adapter in (c) comprises random sequence extension or polymerase chain reaction (PCR).

26. The method of any one of claims 1-25, wherein the RNA primer comprises a poly T tail sequence.

27. The method of any one of claims 1-27, wherein the RNA primer comprises a random sequence.

28. The method of any one of claims 1-27, wherein the RNA primer and/or a DNA primer comprises a barcode sequence for distinguishing cells.

29. The method of claim 28, wherein a DNA barcode is assigned to a DNA primer by tagmentation, PCR, or a combination of tagmentation and PCR.

30. The method of claim 28 or 29, wherein a DNA barcode is assigned to a DNA primer by ligation, tagmentation, PCR, or a combination of two or more of ligation, tagmentation and PCR.

31. The method of any one of claims 28-30, wherein a RNA barcode is assigned to a RNA primer by reverse transcription, PCR, or the combination of reverse transcription, ligation and PCR.

32. The method of any one of claims 28-31, wherein the DNA primer and/or the RNA primer comprise two, three, four, or more different barcode sequences.

33. The method of claim 32, wherein the two or more different barcode sequences are assigned at the 3′ ends, the 5′ ends, or in a combination of the 3′ and 5′ ends of the DNA or RNA.

34. The method of any one of claims 28-33, wherein the DNA primer and/or RNA primer comprises a unique molecular identifier (UMI).

35. The method of any one of claims 28-34, wherein the RNA primer comprises a modification and/or a label.

36. The method of claim 35, wherein the label is a detectable label.

37. The method of any one of claims 35-36, wherein the modification, label, or detectable label comprises a biotin modification used to separate the cDNA from the DNA.

38. The method of any one of claims 1-37, wherein template switch oligonucleotides (TSOs) are added during (d) to form a second end of the cDNA-adaptor molecule.

39. The method of any one of claims 1-38, wherein the co-amplification of the DNA and RNA comprises a polymerase chain reaction (PCR).

40. The method of any one of claims 1-39, wherein the same reaction compartment comprises a test tube, a well, a micro-well, a nano-well or a chip array.

41. The method of any one of claims 1-40, wherein the same reaction compartment comprises a plurality of reaction compartments, optionally a plurality of test tubes, a plurality of wells, a plurality of micro-wells, a plurality of nano-wells or a plurality of chip arrays.

42. The method of claim 41, wherein the plurality of reaction compartments comprising the plurality of DNA amplicons and the plurality of cDNA amplicons are pooled.

43. The method of any one of claims 1-42, further comprising (f) separating the plurality of DNA amplicons from the plurality of cDNA amplicons after co-amplification.

44. The method of claim 43, wherein the plurality of DNA amplicons are separated from the plurality of cDNA amplicons using fragment size, biotin labels, or adapter sequence features.

45. The method of claim 43 or 44, further comprising (g) sequencing the plurality of DNA amplicons and the plurality of cDNA amplicons.

46. The method of claim 45, wherein the sequencing comprises paired-end sequencing or single-read sequencing.

47. The method of claim 45, wherein the sequencing comprises next-generation sequencing (NGS), single molecule sequencing, or nanopore sequencing.

48. The method of any one of claims 45-47, further comprising identifying a mutation in the DNA or RNA.

49. The method of claim 48, wherein the mutation is an insertion, a deletion, or a substitution.

50. The method of claim 49, wherein the mutation is a single nucleotide variation.

51. The method of claim 49, wherein the mutation is a structural variant.

52. The method of any one of claims 48-50, wherein the mutation is associated with a phenotype of interest.

53. The method of any one of claims 43-52, comprising detecting genomic copy number variation.

54. The method of any one of claims 43-53, further comprising performing transcriptome quantification or isoform analysis.

55. The method of any one of claims 1-54, wherein the method comprises production of CDNA.

56. The method of any one of claims 1-55, wherein the method comprises production of a cDNA library.

57. The method of claim 56, wherein the cDNA library is used to prepare a 3′ RNA-seq library.
